(19) United States
(12) Patent Application Publication
(10) Pub. No.: US 2025/0345213 A1
(43) Pub. Date: Nov. 13, 2025

(54) METHODS AND APPARATUSES FOR MAKING ABSORBENT ARTICLES WITH ELASTOMERIC LAMINATES HAVING BEEN ACCUMULATED AND REFRESHED

(71) Applicant: The Procter & Gamble Company, Cincinnati, OH (US)

(72) Inventors: Tina BROWN, Cincinnati, OH (US); Michael Devin LONG, Harrison Twp, OH (US); Abhishek SURUSHE, Mason, OH (US); Koichi MORIMOTO, Cincinnati, OH (US)

(21) Appl. No.: 19/192,457

(22) Filed: Apr. 29, 2025

Related U.S. Application Data
(60) Provisional application No. 63/643,629, filed on May 7, 2024.

(51) Int. Cl. A61F 13/49; A61F 13/494

(52) U.S. Cl. CPC A61F 13/49061; A61F 13/49426; A61F 2013/4905; A61F 2013/4944

(57) ABSTRACT

The present disclosure relates to methods for manufacturing absorbent articles, and in particular, to methods for making elastomeric laminates that may be accumulated and subsequently refreshed to be used as components of absorbent articles. In some configurations, the elastomeric laminate may be wound onto a roll and may comprise elastic strands bonded in a stretched state with a first substrate, wherein the first substrate on the roll comprises a roll substrate strain value that is greater than zero. During the process of assembling absorbent articles, the elastomeric laminate may be unwound from the roll and first discrete contiguous connections between the strands and the first substrate may then be fractured and disrupted to form second discrete contiguous connections by applying localized tensions to the elastomeric laminate.

### CROSS REFERENCE TO RELATED APPLICATION

This application claims the benefit of U.S. Provisional Application No. 63/643,629, filed May 7, 2024, which is incorporated by reference herein in its entirety.

### FIELD

The present disclosure relates to methods for manufacturing absorbent articles, and more particularly, to apparatuses and methods for making absorbent articles with elastomeric laminates that have been accumulated and subsequently refreshed.

### BACKGROUND

Along an assembly line, various types of articles, such as for example, diapers and other absorbent articles, may be assembled by adding components to and/or otherwise modifying an advancing, continuous web of material. For example, in some processes, advancing webs of material are combined with other advancing webs of material. In other examples, individual components created from advancing webs of material are combined with advancing webs of material, which in turn, are then combined with other advancing webs of material. In some cases, individual components created from an advancing web or webs are combined with other individual components created from other advancing webs. Webs of material and component parts used to manufacture diapers may include: backsheets, topsheets, leg cuffs, waist bands, absorbent core components, front and/or back ears, fastening components, and various types of elastic webs and components such as leg elastics, barrier leg cuff elastics, stretch side panels, and waist elastics. Once the desired component parts are assembled, the advancing web(s) and component parts are subjected to a final knife cut to separate the web(s) into discrete diapers or other absorbent articles.

Some absorbent articles have components that include elastomeric laminates. Such elastomeric laminates may include an elastic material bonded to one or more nonwovens. The elastic material may include an elastic film and/or elastic strands. In some laminates, a plurality of elastic strands are joined between two substrates, such as nonwovens, while the plurality of strands are in a stretched condition so that when the elastic strands relax, the nonwovens gather between the locations where the nonwovens are bonded to the elastic strands, and in turn, forms corrugations. The resulting elastomeric laminate may be extendable to the extent that the corrugations allow the elastic strands to elongate.

Some elastomeric laminate material constructions utilizing nonwovens and stranded elastics may be complex and space-consuming to manufacture. As such, it may be beneficial in some cases to assemble, gather, and store elastomeric laminates in a facility or operation separate from absorbent article converting or manufacturing lines to help optimize the converting operations.

For example, some absorbent article assembly processes may be configured to utilize elastomeric laminates comprising relatively large numbers of individual elastic strands. Assembling such elastomeric laminates can be a delicate and complex process requiring a relatively large manufacturing area or machine footprint. As such, assembling elastomeric laminates “online” or simultaneous with or as part of an absorbent article assembly process may increase the complexity of such assembly processes. Thus, in order to reduce machine footprint, converting operation complexity, and/or delivered cost, some absorbent article assembly processes may be adapted to utilize elastomeric laminates that have been manufactured “offline” with a process and/or machine wherein elastic strands have been laminated between nonwovens separate from an absorbent article converter. In turn, the elastomeric laminate may be accumulated, such as for example, by being wound onto a roll. The accumulated elastomeric laminate may be stored and/or moved to a location for incorporation into an absorbent article manufacturing process, wherein the elastomeric laminate may be unwound from the roll and converted into an absorbent article component.

In some operations, an elastomeric laminate may be wound on a roll in a fully extended condition in order to increase the storage efficiency of the elastomeric laminate on the roll. However, storing the elastomeric laminate in such a fully extended condition on a roll may result in degraded stretch properties. For example, when assembling elastomeric laminates, glue may be applied to elastic strands and/or nonwoven substrates. In offline making operations where the elastomeric laminate is stored at full extension, the glue may crystallize or set such that the elastic strands and nonwovens may be glued together along relatively large lengths of the elastic strands, and in turn, may compromise stretch engine performance of the elastomeric laminate. This phenomenon may also be exacerbated by a relatively large winding compression of the elastomeric laminate on the roll. As a result, when the elastomeric laminate is removed from the roll, the nonwovens resist contraction of the elastic strands along the glued lengths, which may result in reduced rugosity and/or gather formation.

Some steps may be taken in an attempt to overcome compromised stretch performance resulting from offline elastomeric laminate assembly operations where the elastomeric laminate is wound onto a roll in a fully extended state. For example, some operations may be adapted to store or gather elastomeric laminates at a relatively low density, such as loosely wound on rolls or lightly packed in boxes. As such, the glue may crystallize or set in the contracted condition and the elastics and nonwovens may be glued together along relatively small lengths of the elastic strands. As a result, when the elastomeric laminate is removed from the roll, the nonwovens resist contraction of the elastic strands along the relatively small glued lengths, which in turn, may mitigate loss of rugosity and/or gather formation. However, storing an elastomeric laminate in a contracted state on a roll provides relatively shorter linear lengths of the elastomeric laminate on a single roll, thus decreasing production time for each roll and increasing change-over time/effort for replacing rolls of material at the absorbent article converting operation due to shorter distances of total laminate being stored on each roll.

Consequently, it would be beneficial to provide methods and apparatuses for making, storing, and converting offline-made elastomeric laminates constructed with stranded elastics and adhesives where the storage state enables the nonwoven substrates to be retained at or near full extension or with strain values that are above zero for optimized converting efficiency, while at the same time mitigating compromised stretch performance in the final converted product.

### SUMMARY

In one form, a method for assembling absorbent articles comprises steps of: unwinding an elastomeric laminate from a roll, wherein the elastomeric laminate comprises elastic strands bonded in a stretched state with a first substrate, wherein the first substrate on the roll comprises a roll substrate strain value that is greater than zero, the elastomeric laminate further comprising first discrete contiguous connections between the clastic strands and the first substrate formed by adhesive at an ambient temperature positioned between the elastic strands and the first substrate, the first discrete contiguous connections comprising first lengths extending along a direction of stretch of the clastic strands; advancing the elastomeric laminate from the roll in a machine direction, wherein the elastic strands are separated from each other in a cross direction; disrupting the first discrete contiguous connections to form second discrete contiguous connections by applying localized tensions to the elastomeric laminate to stretch the elastomeric laminate in discrete locations such that strain values in the first substrate in the discrete locations increase from a first substrate strain value to a second substrate strain value that is greater than the first substrate strain value, and wherein the second discrete contiguous connections comprise second lengths, wherein the second lengths are less than the first lengths, and removing the localized tensions from the elastomeric laminate to provide a refreshed elastomeric laminate; and converting the refreshed elastomeric laminate into an absorbent article component.

In another form, a method for supplying an elastomeric laminate comprises steps of: advancing a first substrate in a machine direction; unwinding elastic strands from spools and advancing the elastic strands in the machine direction; separating neighboring elastic strands at a first distance from each other in a cross direction; stretching the elastic strands in the machine direction; applying adhesive to at least one of the elastic strands and the first substrate, wherein the adhesive is applied at an application temperature above an ambient temperature; forming an elastomeric laminate by combining the stretched elastic strands with the first substrate; stretching the first substrate to a strain value that is greater than zero; winding the elastomeric laminate onto the roll with the first substrate having the strain value that is greater than zero; and allowing the adhesive in the elastomeric laminate on the roll to cool to the ambient temperature and form first discrete contiguous connections between the elastic strands and the first substrate formed by the adhesive at the ambient temperature positioned between the elastic strands and the first substrate between the elastic strands and the first substrate, the first discrete contiguous connections comprising first lengths extending along a direction of stretch of the elastic strands.

### BRIEF DESCRIPTION OF THE DRAWINGS

FIG. 1A is a front perspective view of a diaper pant that may include elastomeric laminates in accordance with the present disclosure.

FIG. 1B is a rear perspective view of a diaper pant that may include elastomeric laminates in accordance with the present disclosure.

FIG. 2 is a partially cut away plan view of the diaper pant shown in FIGS. 1A and 1B in a flat, uncontracted state.

FIG. 3A is a cross-sectional view of the diaper pant of FIG. 2 taken along line 3A-3A.

FIG. 3B is a cross-sectional view of the diaper pant of FIG. 2 taken along line 3B-3B.

FIG. 4A is a plan view of an absorbent article in the form of a taped diaper that may include elastomeric laminates in accordance with the present disclosure with the portion of the diaper that faces away from a wearer oriented towards the viewer.

FIG. 4B is a plan view of the absorbent article of FIG. 4A that may include elastomeric laminates in accordance with the present disclosure with the portion of the diaper that faces toward a wearer oriented towards the viewer.

FIG. 5 is a schematic side view of a converting apparatus adapted to manufacture an elastomeric laminate including a plurality of elastic strands positioned between a first substrate and a second substrate, wherein the assembled elastomeric laminate is wound onto a roll.

FIG. 6 is a view of the converting apparatus of FIG. 5 taken along line 6-6.

FIG. 7 is an isometric view of a spool of an elastic strand wound onto a core.

FIG. 8 is a front side view of an unwinder.

FIG. 9 is a front side view of a strand guide.

FIG. 10 is a front side view of a front side view of an unwinder configured as a surface driven unwinder.

FIG. 11 is a view of the unwinder of FIG. 10 taken along line 11-11.

FIG. 12 shows an example of an empty beam having two side plates connected with opposing end portions of a mandrel core.

FIG. 13 shows an example of the beam of FIG. 12 with a plurality of strands wound thereon.

FIG. 14 is a schematic side view of an apparatus adapted to unwind an elastomeric laminate from a roll and refresh the elastomeric laminate before advancing to an absorbent article assembly process.

FIGS. 15 and 16 illustrate schematic representations of assembly transformations that may be utilized to assemble diaper pants from refreshed elastomeric laminates.

FIG. 17 is a schematic cross section view of an elastomeric laminate illustrating adhesive that has been applied during the laminate making process.

FIG. 18 is a detailed schematic cross section view of an elastomeric laminate illustrating compressive forces acting on the elastomeric laminate that cause warm adhesive to deform and progress lengthwise along the direction of stretch of the elastic strands.

FIG. 19 is a cross sectional view of the elastomeric laminate illustrating first discrete contiguous connections extending along a direction of stretch of the elastic strands.

FIG. 20A illustrates a cross-sectional view of an elastomeric laminate in a fully extended state wherein the first substrate and the second substrate are bonded with an elastic strand in a stretched state with first discrete contiguous connections having first lengths L1.

FIG. 20B illustrates a cross-sectional view of the elastomeric laminate of FIG. 20A when the elastic strand is allowed to contract.

FIG. 21A illustrates a cross-sectional view of a refreshed elastomeric laminate in a fully extended state wherein the first substrate and the second substrate are bonded with an elastic strand in a stretched state with second discrete contiguous connections having second lengths L2.

FIG. 21B illustrates a cross-sectional view of the refreshed elastomeric laminate of FIG. 21A when the elastic strand is allowed to contract.

FIG. 22 shows a refreshing apparatus comprising a first web metering device and a second web metering device.

FIG. 23 shows a refreshing apparatus comprising a first gear and a second gear.

FIG. 23A is a detailed view of an elastomeric laminate advancing between intermeshing gear teeth from FIG. 23.

FIG. 24 shows a refreshing apparatus comprising a ring rolling apparatus.

### DETAILED DESCRIPTION

The following term explanations may be useful in understanding the present disclosure:

“Absorbent article” is used herein to refer to consumer products whose primary function is to absorb and retain soils and wastes. Absorbent articles can comprise sanitary napkins, tampons, panty liners, interlabial devices, wound dressings, wipes, disposable diapers including taped diapers and diaper pants, inserts for diapers with a reusable outer cover, adult incontinent diapers, adult incontinent pads, and adult incontinent pants. The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

An “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

The term “substrate” is used herein to describe a material which is primarily two-dimensional (i.e., in an XY plane) and whose thickness (in a Z direction) is relatively small (i.e., 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. Nonwovens do not have a woven or knitted filament pattern.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

The term “taped diaper” (also referred to as “open diaper”) refers to disposable absorbent articles having an initial front waist region and an initial back waist region that are not fastened, pre-fastened, or connected to each other as packaged, prior to being applied to the wearer. A taped diaper may be folded about the lateral centerline with the interior of one waist region in surface to surface contact with the interior of the opposing waist region without fastening or joining the waist regions together. Example taped diapers are disclosed in various suitable configurations U.S. Pat. Nos. 5,167,897, 5,360,420, 5,599,335, 5,643,588, 5,674,216, 5,702,551, 5,968,025, 6,107,537, 6,118,041, 6,153,209, 6,410,129, 6,426,444, 6,586,652, 6,627,787, 6,617,016, 6,825,393, and 6,861,571; and U.S. Patent Publication Nos. 2013/0072887 A1; 2013/0211356 A1; and 2013/0306226 A1, all of which are incorporated by reference herein.

The term “pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be preformed or pre-fastened by various techniques including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened or seamed, front waist fastened or seamed, rear waist fastened or seamed). Example diaper pants in various configurations are disclosed in U.S. Pat. Nos. 4,940,464; 5,092,861; 5,246,433; 5,569,234; 5,897,545; 5,957,908; 6,120,487; 6,120,489; 7,569,039 and U.S. Patent Publication Nos. 2003/0233082 A1; 2005/0107764 A1, 2012/0061016 A1, 2012/0061015 A1; 2013/0255861 A1; 2013/0255862 A1; 2013/0255863 A1; 2013/0255864 A1; and 2013/0255865 A1, all of which are incorporated by reference herein.

The present disclosure relates to methods for manufacturing absorbent articles, and in particular, to methods for making elastomeric laminates that may be accumulated and subsequently refreshed to be used as components of absorbent articles. In some configurations, the elastomeric laminate may be wound onto a roll and may include a first substrate, a second substrate, and elastic material positioned between the first substrate and second substrate. For example, the elastomeric laminate may comprise elastic strands bonded in a stretched state with a first substrate, wherein the first substrate on the roll comprises a roll substrate strain value that is greater than zero. The elastomeric laminate may further comprise first discrete contiguous connections between the clastic strands and the first substrate formed by adhesive at an ambient temperature positioned between the clastic strands and the first substrate. The first discrete contiguous connections may comprise first lengths extending along a direction of stretch of the elastic strands. During the process of assembling absorbent articles, the elastomeric laminate may be unwound from the roll and may advance from the roll in a machine direction, wherein the clastic strands are separated from each other in a cross direction. The first discrete contiguous connections may then be fractured and disrupted to form second discrete contiguous connections by applying localized tensions to the elastomeric laminate to stretch the elastomeric laminate in discrete locations such that strain values in the first substrate in the discrete locations increase from a first substrate strain value to a second substrate strain value that is greater than the first substrate strain value. And the second discrete contiguous connections may comprise second lengths, wherein the second lengths are less than the first lengths. The localized tensions are removed from the elastomeric laminate to provide a refreshed elastomeric laminate. The refreshed elastomeric laminate is then converted into an absorbent article component.

In some configurations of a method for supplying an elastomeric laminate, a first substrate may be advanced in a machine direction; and elastic strands may be unwound from spools and advanced in the machine direction. Neighboring elastic strands are separated from each other in a cross direction and stretched in the machine direction. Adhesive may be applied to at least one of the elastic strands and the first substrate, wherein the adhesive is applied at an application temperature above an ambient temperature. The elastomeric laminate may be formed by combining the stretched elastic strands with the first substrate. The first substrate of the elastomeric laminate may be stretched to a strain value that is greater than zero; and the elastomeric laminate may be accumulated, such as being wound onto a roll, with the first substrate having the roll substrate strain value that is greater than zero. The adhesive in the elastomeric laminate on the roll is allowed to cool to the ambient temperature and form first discrete contiguous connections between the elastic strands and the first substrate formed by the adhesive at the ambient temperature positioned between the clastic strands and the first substrate between the elastic strands and the first substrate. In some configurations, the roll substrate strain value of the first substrate is decreased during the winding process as a diameter of the roll increases, while also maintaining the roll substrate strain value above zero. In turn, the accumulated elastomeric laminate may be stored and/or moved to a location for incorporation into a manufacturing process, such as an absorbent article assembly process, wherein the elastomeric laminate may be converted into an absorbent article component.

It is to be appreciated that the elastomeric laminates made according to the processes and apparatuses discussed herein may be used to construct various types of components used in the manufacture of different types of absorbent articles, such as diaper pants and taped diapers. To help provide additional context to the subsequent discussion of the process embodiments, the following provides a general description of absorbent articles in the form of diapers that include components including the elastomeric laminates that may be produced with the methods and apparatuses disclosed herein.

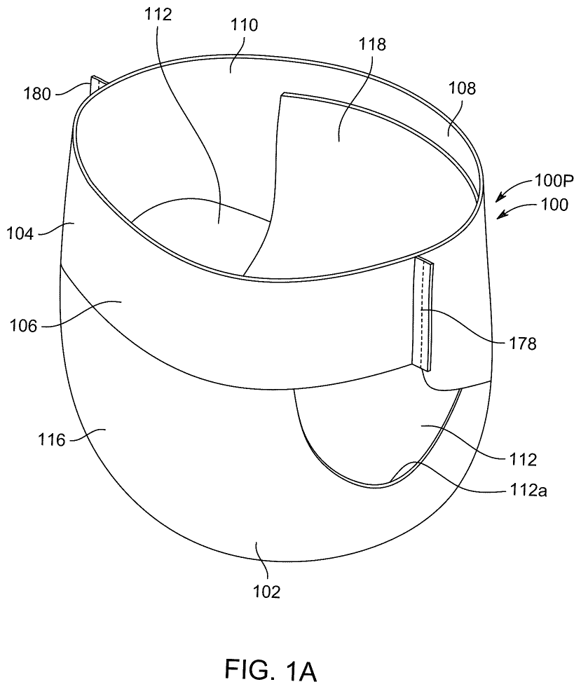

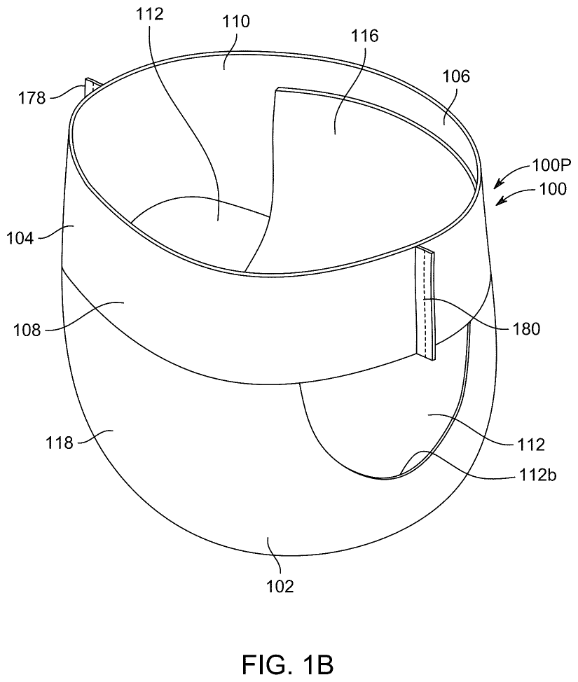

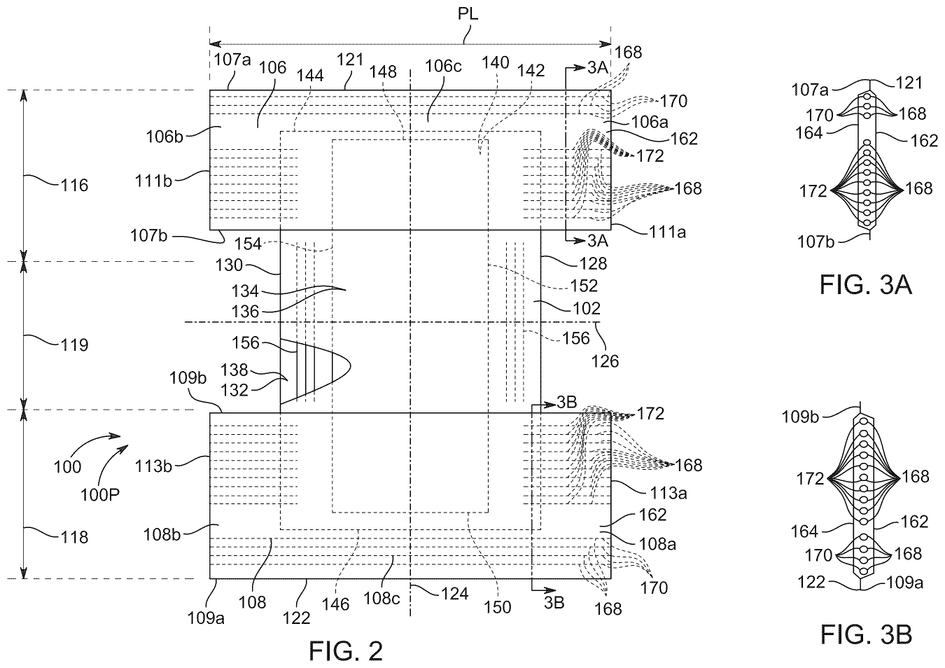

For the purposes of a specific illustration, FIGS. 1A, 1B, and 2 show an example of an absorbent article 100 in the form of a diaper pant 100P that may include components constructed from elastomeric laminates assembled in accordance with the apparatuses and methods disclosed herein. In particular, FIGS. 1A and 1B show perspective views of a diaper pant 100P in a pre-fastened configuration, and FIG. 2 shows a plan view of the diaper pant 100P with the portion of the diaper that faces away from a wearer oriented toward the viewer. The diaper pant 100P includes a chassis 102 and a ring-like elastic belt 104. As discussed below in more detail, a first elastic belt 106 and a second elastic belt 108 are bonded together to form the ring-like elastic belt 104.

With continued reference to FIG. 2, the diaper pant 100P and the chassis 102 each include a first waist region 116, a second waist region 118, and a crotch region 119 disposed intermediate the first and second waist regions. The first waist region 116 may be configured as a front waist region, and the second waist region 118 may be configured as back waist region. The diaper 100P may also include a laterally extending front waist edge 121 in the front waist region 116 and a longitudinally opposing and laterally extending back waist edge 122 in the back waist region 118. To provide a frame of reference for the present discussion, the diaper 100P and chassis 102 of FIG. 2 are shown with a longitudinal axis 124 and a lateral axis 126. In some embodiments, the longitudinal axis 124 may extend through the front waist edge 121 and through the back waist edge 122. And the lateral axis 126 may extend through a first longitudinal or right side edge 128 and through a midpoint of a second longitudinal or left side edge 130 of the chassis 102.

As shown in FIGS. 1A, 1B, and 2, the diaper pant 100P may include an inner, body facing surface 132, and an outer, garment facing surface 134. The chassis 102 may include a backsheet 136 and a topsheet 138. The chassis 102 may also include an absorbent assembly 140, including an absorbent core 142, disposed between a portion of the topsheet 138 and the backsheet 136. As discussed in more detail below, the diaper 100P may also include other features, such as leg elastics and/or leg cuffs to enhance the fit around the legs of the wearer.

As shown in FIG. 2, the periphery of the chassis 102 may be defined by the first longitudinal side edge 128, a second longitudinal side edge 130, a first laterally extending end edge 144 disposed in the first waist region 116, and a second laterally extending end edge 146 disposed in the second waist region 118. Both side edges 128 and 130 extend longitudinally between the first end edge 144 and the second end edge 146. As shown in FIG. 2, the laterally extending end edges 144 and 146 are located longitudinally inward from the laterally extending front waist edge 121 in the front waist region 116 and the laterally extending back waist edge 122 in the back waist region 118. When the diaper pant 100P is worn on the lower torso of a wearer, the front waist edge 121 and the back waist edge 122 may encircle a portion of the waist of the wearer. At the same time, the side edges 128 and 130 may encircle at least a portion of the legs of the wearer. And the crotch region 119 may be generally positioned between the legs of the wearer with the absorbent core 142 extending from the front waist region 116 through the crotch region 119 to the back waist region 118.

As previously mentioned, the chassis 102 may include a backsheet 136. The backsheet 136 may also define a portion of the outer, garment facing surface 134 of the chassis 102. The backsheet 136 is generally that portion of the absorbent article 100 positioned proximate to the garment-facing surface of the absorbent assembly 140. The backsheet 136 may be joined to portions of the topsheet 138, the absorbent assembly 140, and/or any other layers of the absorbent article by any attachment methods known to those of skill in the art. The backsheet 136 prevents, or at least inhibits, the bodily exudates absorbed and contained in the absorbent core 142 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials, such as films, which permit vapors to escape from the absorbent article, while still preventing, or at least inhibiting, bodily exudates from passing through the backsheet. The backsheet 136 may also comprise a woven or nonwoven material, polymeric films such as thermoplastic films of polyethylene or polypropylene, and/or a multi-layer or composite materials comprising a film and a nonwoven material, such as having an inner film layer and an outer nonwoven layer or outer cover material. The outer cover material (sometimes referred to as a backsheet nonwoven) may comprise one or more nonwoven materials joined to a film. The outer cover material may form at least a portion of the garment facing surface 134 of the backsheet 136 so that film is not present on the garment facing surface 134. The outer cover material may comprise a bond pattern, apertures, and/or three-dimensional features. The outer cover material may be a hydroentangled nonwoven material or a variable basis weight nonwoven material. Such a material may have one or more layers.

Also described above, the diaper 100 may include a topsheet 138. The topsheet 138 may also define all or part of the inner, wearer facing surface 132 of the chassis 102. The topsheet 138 may be compliant, soft feeling, and non-irritating to the wearer's skin. It may be elastically stretchable in one or two directions. Further, the topsheet 138 may be liquid pervious, permitting liquids (e.g., menses, urine, and/or runny feces) to penetrate through its thickness. A topsheet 138 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art.

Topsheets 138 may be selected from high loft nonwoven topsheets, apertured film topsheets and apertured nonwoven topsheets. Apertured film topsheets may be pervious to bodily exudates, yet substantially non-absorbent, and have a reduced tendency to allow fluids to pass back through and rewet the wearer's skin. Exemplary apertured films may include those described in U.S. Pat. Nos. 5,628,097; 5,916,661; 6,545,197; and 6,107,539, all of which are incorporated by reference herein.

As mentioned above, the diaper 100 may also include an absorbent assembly 140 that is joined to the chassis 102. As shown in FIGS. 1A and 1B, the absorbent assembly 140 may have a laterally extending front edge 148 in the front waist region 116 and may have a longitudinally opposing and laterally extending back edge 150 in the back waist region 118. The absorbent assembly may have a longitudinally extending right side edge 152 and may have a laterally opposing and longitudinally extending left side edge 154, both absorbent assembly side edges 152 and 154 may extend longitudinally between the front edge 148 and the back edge 150. The absorbent assembly 140 may additionally include one or more absorbent cores 142 or absorbent core layers. The absorbent core 142 may be at least partially disposed between the topsheet 138 and the backsheet 136 and may be formed in various sizes and shapes that are compatible with the diaper. Exemplary absorbent structures for use as the absorbent core of the present disclosure are described in U.S. Pat. Nos. 4,610,678; 4,673,402; 4,888,231; and 4,834,735, all of which are incorporated by reference herein.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprise primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 A1 and 2004/0097895 A1, all of which are incorporated by reference herein.

In some configurations, the absorbent assembly 140 may include an acquisition system disposed between the topsheet 138 and a wearer facing side of the absorbent core 142. The acquisition system may be in direct contact with the absorbent core 142 and may comprise a single layer or multiple layers, such as an upper acquisition layer (also referred to herein as a first acquisition layer) facing towards the wearer's skin and a lower acquisition layer (also referred to herein as a second acquisition layer) facing the garment of the wearer. In some embodiments, the acquisition system may function to receive a surge of liquid, such as a gush of urine. As such, the acquisition system may serve as a temporary reservoir for liquid until the absorbent core 142 can absorb the liquid. Exemplary acquisition systems and associated manufacturing processes are described in U.S. Pat. Nos. 8,603,277 and 8,568,566; U.S. Patent Publication Nos. 2012/0316046 A1 and 2014/0163504 A1, all of which are hereby incorporated by reference herein.

As previously mentioned, the diaper 100P may also include elasticized leg cuffs 156. It is to be appreciated that the leg cuffs 156 can be and are sometimes also referred to as leg bands, side flaps, barrier cuffs, elastic cuffs or gasketing cuffs. The elasticized leg cuffs 156 may be configured in various ways to help reduce the leakage of body exudates in the leg regions. Example leg cuffs 156 may include those described in U.S. Pat. Nos. 3,860,003; 4,909,803; 4,695,278; 4,795,454; 4,704,115; 4,909,803; and U.S. Patent Publication No. 2009/0312730 A1, all of which are incorporated by reference herein.

As mentioned above, diaper pants may be manufactured with a ring-like clastic belt 104 and provided to consumers in a configuration wherein the front waist region 116 and the back waist region 118 are connected to each other as packaged, prior to being applied to the wearer. The ring-like clastic belt may be formed by joining a first elastic belt to a second elastic belt with a permanent side scam or with an openable and reclosable fastening system disposed at or adjacent the laterally opposing sides of the belts. As such, diaper pants may have a continuous perimeter waist opening 110 and continuous perimeter leg openings 112 such as shown in FIGS. 1A and 1B. Example clastic belt 170 configurations are disclosed in U.S. Patent Publication Nos. 2018/0168878 A1; 2018/0168877 A1; 2018/0168880 A1; 2018/0170027 A1; 2018/0169964 A1; 2018/0168879 A1; 2018/0170026 A1; 2018/0168889 A1; 2018/0168874 A1; 2018/0168875 A1; 2018/0168890 A1; 2018/0168887 A1; 2018/0168892 A1; 2018/0168876 A1; 2018/0168891 A1; 2019/0070042 A1; 2019/0070041 A1; 2021/0282797 A1; 2021/0275362 A1; 2022/0142828 A1; 2022/0362068 A1; 2024/0000619 A1; 2024/0000630 A1; 2024/0000631 A1; 2024/0000625 A1; 2024/0000624 A1; 2024/0000633 A1; 2024/0000632 A1; 2024/0000626 A1; 2024/0000634 A1; 2024/0000635 A1; 2024/0000637 A1; 2024/0000638 A1; and 2024/0000627 A1, which are all incorporated by reference herein.

As previously mentioned, the ring-like elastic belt 104 may be defined by a first elastic belt 106 connected with a second clastic belt 108. As shown in FIG. 2, the first elastic belt 106 extends between a first longitudinal side edge 111a and a second longitudinal side edge 111b and defines first and second opposing end regions 106a, 106b and a central region 106c. And the second elastic 108 belt extends between a first longitudinal side edge 113a and a second longitudinal side edge 113b and defines first and second opposing end regions 108a, 108b and a central region 108c. The distance between the first longitudinal side edge 111a and the second longitudinal side edge 111b defines the pitch length, PL, of the first elastic belt 106, and the distance between the first longitudinal side edge 113a and the second longitudinal side edge 113b defines the pitch length, PL, of the second clastic belt 108. The central region 106c of the first clastic belt is connected with the first waist region 116 of the chassis 102, and the central region 108c of the second elastic belt 108 is connected with the second waist region 118 of the chassis 102. As shown in FIGS. 1A and 1B, the first end region 106a of the first clastic belt 106 is connected with the first end region 108a of the second clastic belt 108 at first side scam 178, and the second end region 106b of the first clastic belt 106 is connected with the second end region 108b of the second elastic belt 108 at second side seam 180 to define the ring-like clastic belt 104 as well as the waist opening 110 and leg openings 112.

As shown in FIGS. 2, 3A, and 3B, the first clastic belt 106 also defines an outer laterally extending edge 107a and an inner laterally extending edge 107b, and the second clastic belt 108 defines an outer laterally extending edge 109a and an inner laterally extending edge 109b. As such, a perimeter edge 112a of one leg opening may be defined by portions of the inner laterally extending edge 107b of the first elastic belt 106, the inner laterally extending edge 109b of the second clastic belt 108, and the first longitudinal or right side edge 128 of the chassis 102. And a perimeter edge 112b of the other leg opening may be defined by portions of the inner laterally extending edge 107b, the inner laterally extending edge 109b, and the second longitudinal or left side edge 130 of the chassis 102. The outer laterally extending edges 107a, 109a may also define the front waist edge 121 and the laterally extending back waist edge 122 of the diaper pant 100P. The first elastic belt and the second clastic belt may also each include an outer, garment facing layer 162 and an inner, wearer facing layer 164. It is to be appreciated that the first elastic belt 106 and the second clastic belt 108 may comprise the same materials and/or may have the same structure. In some embodiments, the first elastic belt 106 and the second clastic belt may comprise different materials and/or may have different structures. It should also be appreciated that the first clastic belt 106 and the second clastic belt 108 may be constructed from various materials. For example, the first and second belts may be manufactured from materials such as plastic films; apertured plastic films; woven or nonwoven webs of natural materials (e.g., wood or cotton fibers), synthetic fibers (e.g., polyolefins, polyamides, polyester, polyethylene, or polypropylene fibers) or a combination of natural and/or synthetic fibers; or coated woven or nonwoven webs. In some embodiments, the first and second clastic belts include a nonwoven web of synthetic fibers, and may include a stretchable nonwoven. In other embodiments, the first and second clastic belts include an inner hydrophobic, non-stretchable nonwoven material and an outer hydrophobic, non-stretchable nonwoven material.

The first and second clastic belts 106, 108 may also each include belt clastic material interposed between the outer substrate layer 162 and the inner substrate layer 164. The belt elastic material may include one or more clastic elements such as strands, ribbons, films, or panels extending along the lengths of the elastic belts. As shown in FIGS. 2, 3A, and 3B, the belt clastic material may include a plurality of elastic strands 168 which may be referred to herein as outer, waist elastics 170 and inner, waist elastics 172. Elastic strands 168, such as the outer waist elastics 170, may continuously extend laterally between the first and second opposing end regions 106a, 106b of the first elastic belt 106 and between the first and second opposing end regions 108a, 108b of the second clastic belt 108. In some embodiments, some clastic strands 168, such as the inner waist elastics 172, may be configured with discontinuities in areas, such as for example, where the first and second clastic belts 106, 108 overlap the absorbent assembly 140. In some embodiments, the elastic strands 168 may be disposed at a constant interval in the longitudinal direction. In other embodiments, the elastic strands 168 may be disposed at different intervals in the longitudinal direction. The belt elastic material in a stretched condition may be interposed and joined between the uncontracted outer layer and the uncontracted inner layer. When the belt clastic material is relaxed, the belt clastic material returns to an unstretched condition and contracts the outer layer and the inner layer. The belt elastic material may provide a desired variation of contraction force in the area of the ring-like elastic belt. It is to be appreciated that the chassis 102 and elastic belts 106, 108 may be configured in different ways other than as depicted in FIG. 2. The belt elastic material may be joined to the outer and/or inner layers continuously or intermittently along the interface between the belt clastic material and the inner and/or outer belt layers.

In some configurations, the first elastic belt 106 and/or second elastic belt 108 may define curved contours. For example, the inner lateral edges 107b, 109b of the first and/or second clastic belts 106, 108 may include non-linear or curved portions in the first and second opposing end regions. Such curved contours may help define desired shapes to leg opening 112, such as for example, relatively rounded leg openings. In addition to having curved contours, the clastic belts 106, 108 may include clastic strands 168, 172 that extend along non-linear or curved paths that may correspond with the curved contours of the inner lateral edges 107b, 109b.

It is also to be appreciated that the first clastic belt 106 and the second elastic belt 108 may be discrete components that are connected with each other by the chassis 102. In some configurations, the first clastic belt 106 and the second elastic belt 108 may be integrally formed and may be connected with one more outer cover substrates that may extend contiguously from the from the first waist edge 121 to the second waist edge 122.

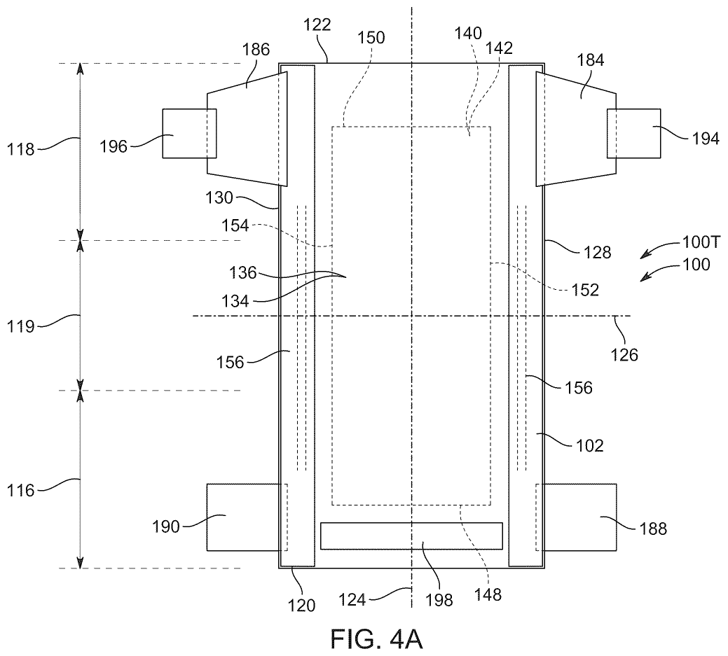

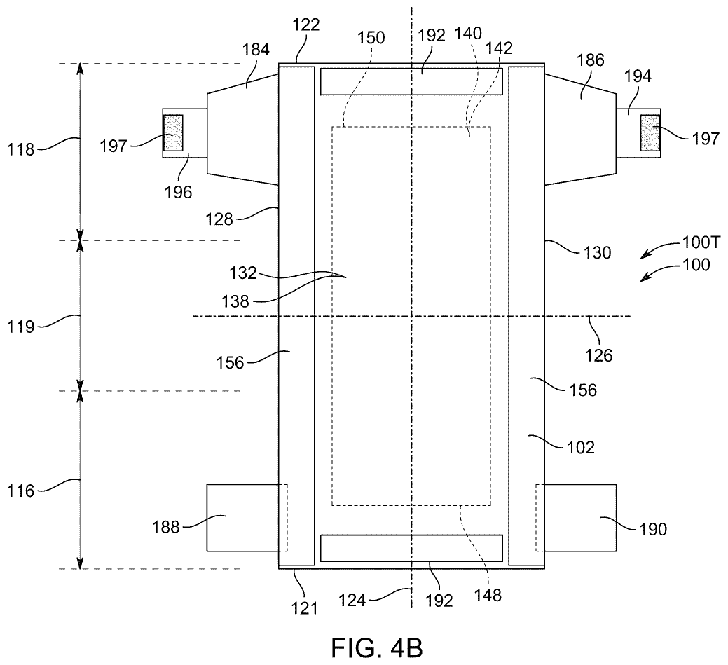

As previously mentioned, absorbent articles 100 may also be configured as taped diapers 100T. For example, FIG. 4A shows a plan view of an absorbent article 100 configured as a taped diaper 100T, with the portion of the diaper that faces away from a wearer oriented towards the viewer. And FIG. 4B shows a plan view of the diaper 100 with the portion of the diaper that faces toward a wearer oriented towards the viewer. The taped diaper 100T shown in FIGS. 4A and 4B may include an absorbent chassis 102 and elasticized leg cuffs 156, such as discussed above. The taped diaper 100T may also include first and second rear side panels 184 and 186; and first and second front side panels 188 and 190.

With continued reference to FIGS. 4A and 4B, when the taped diaper 100T is worn on the lower torso of a wearer, the first waist edge 121 and the second waist edge 122 may encircle a portion of the waist of the wearer. At the same time, the side edges 128 and 130 may encircle at least a portion of the legs of the wearer. And the crotch region 119 may be generally positioned between the legs of the wearer with the absorbent core 142 extending from the front waist region 116 through the crotch region 119 to the back waist region 118.

It is to also be appreciated that a portion or the whole of the diaper 100T may also be made laterally extensible. The additional extensibility may help allow the diaper 100 to conform to the body of a wearer during movement by the wearer. The additional extensibility may also help, for example, the user of the diaper 100, including a chassis 102 having a particular size before extension, to extend the first waist region 116, the second waist region 118, or both waist regions of the diaper 100 and/or chassis 102 to provide additional body coverage for wearers of differing size, i.e., to tailor the diaper to an individual wearer. Such extension of the waist region or regions may give the absorbent article a generally hourglass shape, so long as the crotch region is extended to a relatively lesser degree than the waist region or regions, and may impart a tailored appearance to the article when it is worn.

The taped diaper 100T may also include an elasticized waistband 192. The elasticized waistband 192 may provide improved fit and containment and may be a portion or zone of the diaper 100 that may elastically expand and contract to dynamically fit a wearer's waist. The elasticized waistband 192 may extend longitudinally inwardly from the waist edges 121, 122 of the diaper toward the lateral edges 148, 150 of the absorbent core 142. The diaper 100 may also include more than one elasticized waistband 192, for example, having one waistband 192 positioned in the back waist region 118 and one waistband 192 positioned in the front wait region 116, although other configurations may be constructed with a single elasticized waistband 192. The elasticized waistband 192 may be constructed in a number of different configurations including those described in U.S. Pat. Nos. 4,515,595 and 5,151,092; and U.S. Patent Publication Nos. 2020/0375807 A1; 2020/0375815 A1; 2021/0128366 A1; and 2021/0128369 A1, all of which are incorporated by reference.

Taped diapers 100T may be manufactured and provided to consumers in a configuration wherein the front waist region and the back waist region are not fastened, pre-fastened, or connected to each other as packaged, prior to being applied to the wearer. For example, the taped diaper 100T may be folded about a lateral centerline with the wearer facing surface 132 of the first waist region 116 in surface to surface contact with the wearer facing surface 132 of the second waist region 118 without fastening or joining the waist regions together. The rear side panels 184 and 186 and/or the front side panels 188 and 190 may also be folded laterally inward toward the inner, wearer facing surfaces 132 of the waist regions 116 and 118.

The diaper 100T may also include various configurations of fastening elements to enable fastening of the front waist region 116 and the back waist region 118 together to form a closed waist circumference and leg openings once the diaper is positioned on a wearer. For example, as shown in FIGS. 4A and 4B, the diaper 100 may include first and second fastening members 194, 196, also referred to as tabs, connected with the first and second rear side panels 184, 186, respectively. The diaper may also include first and second front side panels 188, 190, that may or may not include fastening members.

With continued reference to FIGS. 4A and 4B, each side panel 184, 186 and/or fastening member 194, 196 may form a portion of or may be permanently bonded, adhered or otherwise joined directly or indirectly to the chassis 102 laterally inward from the side edge 128 and 130, in one of the front waist region 116 or the back waist region 118. Alternatively, the fastening members 194, 196 may form a portion of or may be permanently bonded, adhered or otherwise joined directly or indirectly to the first and second rear panels 184, 186 at or adjacent the distal edge of the panel and/or the first and second front side panels 188 and 190 at or adjacent the distal edge of the side panel. It is to be appreciated that the fastening members and/or side panels may be assembled in various ways, such as disclosed for example, in U.S. Pat. No. 7,371,302, which is incorporated by reference herein. The fastening members 194, 196 and/or side panels 184, 186, 188, 190 may also be permanently bonded or joined at or adjacent the side edges 128 and 130 of the chassis 102 in various ways, such as for example, by adhesive bonds, sonic bonds, pressure bonds, thermal bonds or combinations thereof, such as disclosed for example, U.S. Pat. No. 5,702,551, which is incorporated by reference herein.

Referring now to FIG. 4B, the first fastening member 194 and/or the second fastening member 196 may include various types of releasably engageable fasteners. The first and second fastening members 194 and/or 196 may also include various types of refastenable fastening structures. For example, the first and second fastening members 194 and 196 may include mechanical fasteners, 197, in the form of hook and loop fasteners, hook and hook fasteners, macrofasteners, buttons, snaps, tab and slot fasteners, tape fasteners, adhesive fasteners, cohesive fasteners, magnetic fasteners, hermaphroditic fasteners, and the like. Some examples of fastening systems and/or fastening members 194, 196 are discussed in U.S. Pat. Nos. 3,848,594; 4,662,875; 4,846,815; 4,894,060; 4,946,527; 5,151,092; 5,221,274; 6,251,097; 6,669,618; 6,432,098; and U.S. Patent Publication Nos. 2007/0078427 A1 and 2007/0093769 A1, all of which are incorporated by reference herein.

As previously mentioned, the fastening members 194 and 196 may be constructed from various materials and may be constructed as a laminate structure. The fastening members 194 and 196 may also be adapted to releasably and/or refastenably engage or connect with another portion of the diaper 100. For example, as shown in FIG. 4A, the diaper 100 may include a connection zone 198, sometimes referred to as a landing zone, in the first waist region 116. As such, when the taped diaper 100 is placed on a wearer, the fastening members 194 and 196 may be pulled around the waist of the wearer and connected with the connection zone 198 in the first waist region 116 to form a closed waist circumference and a pair of laterally opposing leg openings. It is to be appreciated that the connection zone may be constructed from a separate substrate that is connected with the chassis 102 of the taped diaper 100T. In some embodiments, the connection zone may be integrally formed as part of the backsheet 136 of the diaper 100 or may be formed as part of the first and second front panels 188, 190, such as described in U.S. Pat. Nos. 5,735,840 and 5,928,212, all of which are incorporated by reference herein.

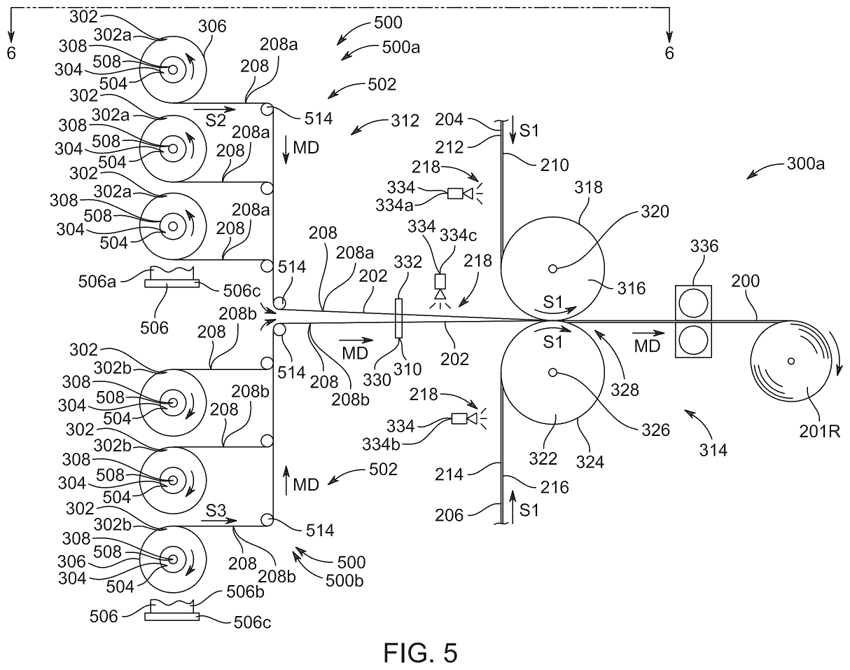

It is to be appreciated that refreshed elastomeric laminates 200R discussed below with reference to FIGS. 5-24 may be used to construct various types of absorbent article components. For example, the refreshed elastomeric laminates 200R may be used as a continuous length of elastomeric belt material that may be converted into the first and second elastic belts 106, 108 discussed above with reference to FIGS. 1A-3B. As such, the elastic material 202 may correspond with the belt elastic material 168 interposed between the outer layer 162 and the inner layer 164, which in turn, may correspond with either the first and/or second substrates 204, 206. In other examples, the refreshed elastomeric laminates 200R may be used to construct waistbands 192 and/or side panels 184, 186 in taped diaper 100T configurations such as discussed above with reference to FIGS. 4A and 4B. In yet other examples, the refreshed elastomeric laminates 200R may be used to construct various types of leg cuff 156, topsheet 138, and/or backsheet configurations. It also to be appreciated that the methods and apparatuses herein may be adapted to operate with various types of absorbent article assembly processes, such as disclosed for example in U.S. Patent Publication Nos. 2013/0255861 A1; 2013/0255862 A1; 2013/0255863 A1; 2013/0255864 A1; and 2013/0255865 A1, which are all incorporated by reference herein.

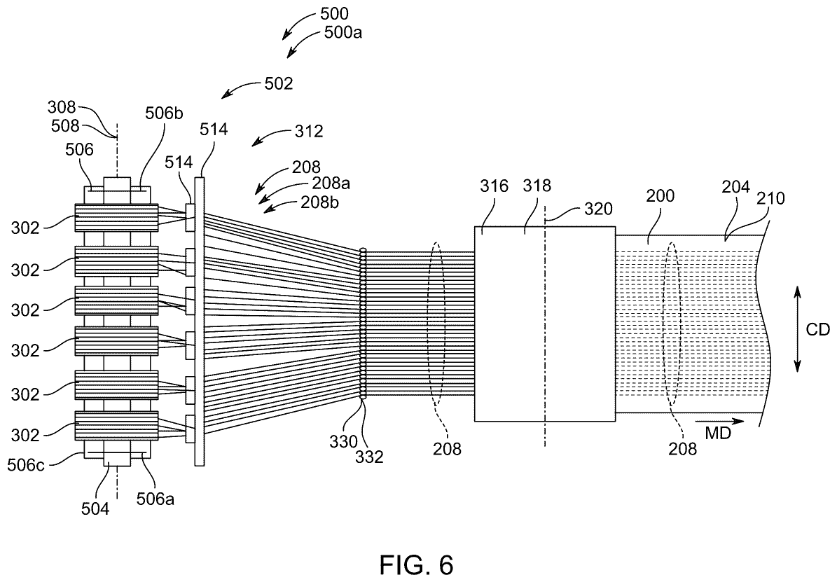

As previously mentioned, apparatuses and methods according to the present disclosure may be utilized to produce elastomeric laminates that may be used to construct various components of diapers, such as elastic belts, waistbands, leg cuffs, side panels, and the like. For example, FIGS. 4 and 5 show schematic views of a converting apparatus 300a adapted to manufacture elastomeric laminates 200. As described in more detail below, the converting apparatus 300a shown in FIGS. 5 and 6 operates to advance a continuous length of clastic material 202, a continuous length of a first substrate 204, and a continuous length of a second substrate 206 along a machine direction MD. It is also to be appreciated that in some configurations, the first substrate and second substrate 204, 206 herein may be defined by two discrete substrates or may be defined by folded portions of a single substrate. The apparatus 300a stretches the clastic material 202 and joins the stretched clastic material 202 with the first and second substrates 204, 206 to produce an elastomeric laminate 200. Although the clastic material 202 is illustrated and referred to herein as elastic strands 208, it is to be appreciated that in some configurations, clastic material 202 may include one or more continuous lengths of clastic strands, ribbons, and/or films. It is also to be appreciated that in some configurations, the elastomeric laminate 200 may include only the first substrate 204 with clastic strands 208 and/or other clastic material 202 bonded thereto.

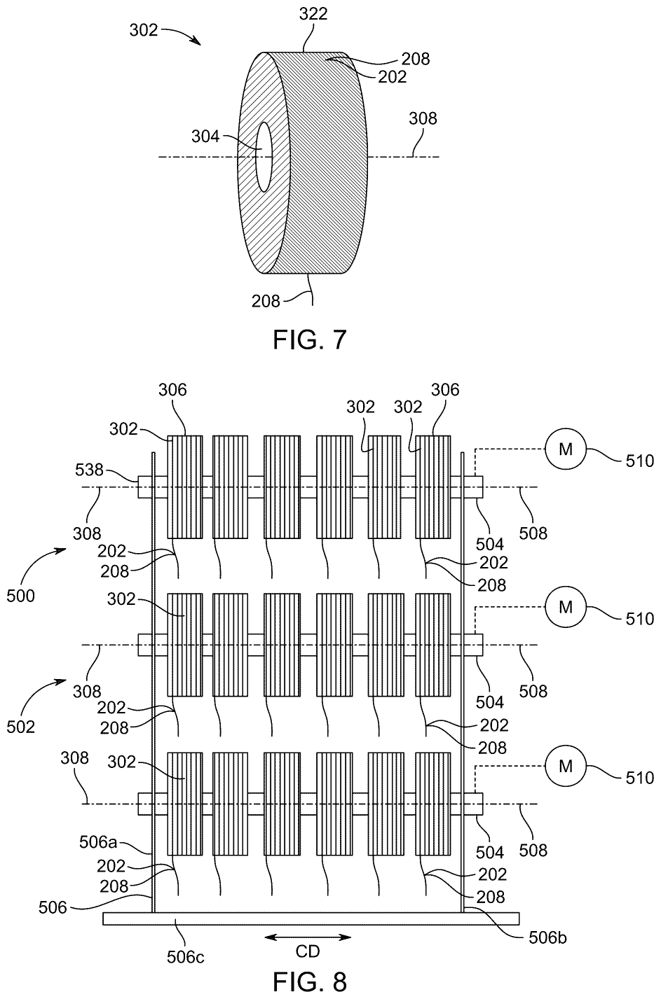

FIGS. 5 and 6 show an example of a converting apparatus 300a that may be configured to assemble elastomeric laminates 200. The apparatus 300a may include a plurality of spools 302 of clastic strands 208. As shown in FIG. 7, each spool 302 may include a single elastic strand 208 wound onto a core 304. The spool 302 may be cylindrically shaped and include an outer circumferential surface 306 defined by the clastic strand 208 wound around the core 304. The spool 302 may also be adapted to rotate about an axis of rotation 308. The core 304 may be cylindrically shaped and the axis of rotation 308 may extend axially through the center of the core 304. With continued reference to FIGS. 5 and 6, the clastic strands 208 are unwound from respective spools 302 by rotating the spools 302 about the cores 304 and/or the axis of rotation 308. The clastic strands 28 advance in a machine direction MD and are combined with the first substrate 204 and the second substrate 206 to form the elastomeric laminate 200.

As shown in FIG. 5, the elastic strands 208 may also advance through a strand guide 310 before being combined with the first substrate 204 and the second substrate 206. As discussed in more detail below, the strand guide 310 spaces or separates neighboring elastic strands 208 from each other at a desired distance in a cross direction CD while being combined with the first substrate 204 and the second substrate 206. The elastic strands 208 may also be stretched in the machine direction MD and combined with the first substrate 204 and the second substrate 206 in the stretched state. The assembled elastomeric laminate 200 may be accumulated, such as for example, by being wound onto a roll 201R. The accumulated elastomeric laminate 200 may be stored and/or moved to a location for incorporation into an absorbent article assembly process wherein the elastomeric laminate 200 may be converted into an absorbent article component. As discussed in more detail below, tension may be maintained on the elastomeric laminate 200 as the elastomeric laminate is accumulated. As such, the elastomeric laminated 200 may be accumulated under tension on a roll for example, stored, and/or moved to a location for incorporation into an absorbent article assembly process. Thus, tension could be maintained on the elastomeric laminate 200 while being unwound and while being incorporated into an absorbent article assembly process, and such tension can be removed from the elastomeric laminate 200 during the assembly process or after the assembly process is complete.

As shown in FIGS. 5 and 6, the converting apparatus 300a for producing an elastomeric laminate 200 may include a first metering device 312 and a second metering device 314. The first metering device 312 may be configured as an unwinder 500 with one or more spools 302 of elastic strands 208 positioned thereon. During operation, the clastic strands 208 advance in the machine direction MD from the unwinder 500 to the second metering device 314. In addition, the clastic strands 208 may be stretched along the machine direction MD while advancing between the unwinder 500 and the second metering device 314. The stretched elastic strands 208 are also joined with the first substrate 204 and the second substrate 206 at the second metering device 314 to produce an elastomeric laminate 200. It is also to be appreciated that the clastic strands 208 may advance along and/or around one or more guide rollers 514. It is to be appreciated that the clastic strands may be stretched along a continuous path while advancing in the machine direction or may be stretched in various steps that provide multiple increases in elongation while advancing in the machine direction.

As shown in FIG. 5, the second metering device 314 includes: a first roller 316 having an outer circumferential surface 318 and rotates about a first axis of rotation 320, and a second roller 322 having an outer circumferential surface 324 and rotates about a second axis of rotation 326. The first roller 316 and the second roller 322 rotate in opposite directions, and the first roller 316 is adjacent the second roller 322 to define a nip 328 between the first roller 316 and the second roller 322. The first roller 316 may rotate such that the outer circumferential surface 318 has a surface speed S1, and the second roller 322 may rotate such that the outer circumferential surface 324 has the same, or substantially the same, surface speed S1.

As shown in FIG. 5, the first substrate 204 includes a first surface 210 and an opposing second surface 212, and the first substrate 204 advances to the first roller 316. In particular, the first substrate 204 advances at speed S1 to the first roller 316 where the first substrate 204 partially wraps around the outer circumferential surface 318 of the first roller 316 and advances through the nip 328. As such, the first surface 210 of the first substrate 204 travels in the same direction as and in contact with the outer circumferential surface 318 of the first roller 316. In addition, the second substrate 206 includes a first surface 214 and an opposing second surface 216, and the second substrate 206 advances to the second roller 322. In particular, the second substrate 206 advances at speed S1 to the second roller 322 where the second substrate 206 partially wraps around the outer circumferential surface 324 of the second roller 322 and advances through the nip 328. As such, the second surface 216 of the second substrate 206 travels in the same direction as and in contact with the outer circumferential surface 324 of the second roller 322. It is to be appreciated that the first and/or substrates 204, 206 may advance at various speeds S1. In some configurations, the first substrate 204 and/or the second substrate 206 may advance at speed S1 from about 150 meters/minute to about 300 meters/minute, specifically reciting all 1 meter/minute increments within the above-recited range and all ranges formed therein or thereby.

With continued reference to FIGS. 5, 6, and 8, the unwinder 500 may include spools 302 of clastic strands 208 wound thereon, wherein each spool 302 is rotatable about a respective axis of rotation 308. As discussed above, the spools 316 may rotate such that the outer circumferential surface 306 of the spools 302 move at a speed S2. As the spools 302 rotate, the clastic strands 208 unwind from the rotating spools 302 and advance at the speed S2 in the machine direction MD to the nip 328. In some configurations, the speed S2 is less than the speed S1, and as such, the clastic strands 208 are stretched in the machine direction MD. In turn, the stretched clastic strands 208 advance through the nip 328 between the first and second substrates 204, 206 such that the clastic strands 208 are joined with the second surface 212 of the first substrate 204 and the first surface 214 of the second substrate 206 to produce a continuous length of elastomeric laminate 200.

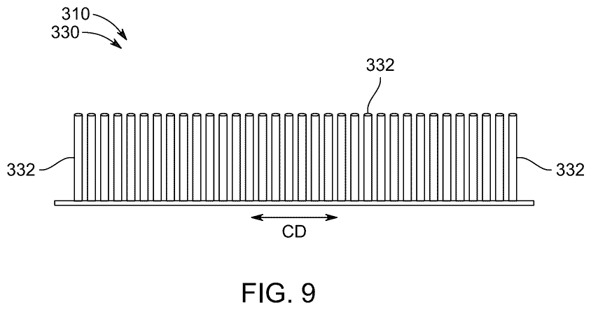

As shown in FIGS. 5 and 9, the elastic strands may advance through a strand guide 310 positioned between the spools 302 and the nip 328. The strand guide 310 may operate to change and/or dictate and/or fix the cross directional CD separation distance between neighboring clastic strands 208 advancing into the nip 328 and in the assembled elastomeric laminate 200. It is to be appreciated that the elastic strands 208 may be separated from each other by various distances in the cross direction CD advancing into the nip 328 and in the assembled elastomeric laminate 200. In some configurations, neighboring elastic strands 208 may be separated from each other by about 0.5 mm to about 4 mm in the cross direction CD, specifically reciting all 0.1 mm increments within the above-recited range and all ranges formed therein or thereby. It is to be appreciated that the strand guide 310 may be configured in various ways. In some configurations, such as shown in FIG. 9, the strand guide 310 may be configured as a comb 330 that may comprise a plurality of tines or reeds 332. In turn, the advancing clastic strands 208 are separated and spaced apart from each other by the tines or reeds 332 in the cross direction CD from each other. In some configurations, the strand guide 310 may include a plurality of rollers that separate and space the clastic strands in the cross direction CD from each other.

As discussed above, it is to be appreciated that the elastomeric laminates 200 assembled herein may include various quantities of elastic strands 208 spaced apart from each other by various distances and may include various decitex values. For example, the elastomeric laminates 200 herein may have various clastic densities, wherein the elastic density may be defined as decitex per elastomeric laminate width. For example, some elastomeric laminates 200 may have an elastic density from about 10 decitex/mm to about 150 decitex/mm, specifically reciting all 1 decitex/mm increments within the above-recited range and all ranges formed therein or thereby. In another example, the elastomeric laminates 200 herein may have various numbers of clastic strands arranged in the cross direction CD per meter of elastomeric laminate cross directional width. For example, some elastomeric laminates 200 may have from about 500 clastic strands/meter of elastomeric laminate width to about 2000 elastic strands/meter of elastomeric laminate width, specifically reciting all 1 elastic strand/meter increments within the above-recited range and all ranges formed therein or thereby.

As shown in FIG. 5, the apparatus 300a may include one or more adhesive applicator devices 334 that may apply adhesive 218 to at least one of the clastic strands 208, the first substrate 204, and the second substrate 206 before being combined to form the elastomeric laminate 200. For example, the first substrate 204 may advance past an adhesive applicator device 334a that applies adhesive 218 to the second surface 212 of the first substrate 204 before advancing to the nip 328. It is to be appreciated that the adhesive 218 may be applied to the first substrate 204 upstream of the first roller 316 and/or while the first substrate 204 is partially wrapped around the outer circumferential surface 318 of the first roller 316. In another example, the second substrate 206 may advance past an adhesive applicator device 334b that applies adhesive 218 to the first surface 214 of the second substrate 206 before advancing to the nip 328. It is to be appreciated that the adhesive 218 may be applied to the second substrate 206 upstream of the second roller 322 and/or while the second substrate 206 is partially wrapped around the outer circumferential surface 324 of the second roller 324. In another example, an adhesive applicator device 334c may be configured to apply adhesive 218 to the elastic strands 208 before and/or while being joined with first substrate 204 and second substrate 206.

It is to be appreciated that the adhesive applicator devices herein 334 be configured in various ways, such as for example, spray nozzles and/or slot coating devices. In some configurations, the adhesive applicator devices 334 may be configured in accordance with the apparatuses and/or methods disclosed in U.S. Pat. Nos. 8,186,296; 9,265,672; 9,248,054; and 9,295,590 and U.S. Patent Publication No. 2014/0148773 A1, all of which are incorporated by reference herein.

As shown in FIG. 5, the apparatus 300a may include a mechanical bonding device 336 that applies the mechanical bonds to the elastomeric laminate 200, such as for example, bonds that may be applied with heat, pressure, and/or ultrasonic devices. Examples of such mechanical bonding devices and methods are disclosed in U.S. Pat. Nos. 4,854,984; 6,291,039; 6,248,195; 8,778,127; and 9,005,392; and U.S. Patent Publication Nos. 2014/0377513 A1; and 2014/0377506 A1, all of which are incorporated by reference herein. It is to be appreciated that the mechanical bonding device 336 may apply mechanical bonds to the elastomeric laminate at or downstream of the nip 328. The mechanical bonding device may apply bonds that bond the first substrate 204, the second substrate 206, and/or elastic strands 208 together and/or may act to trap or immobilize discrete lengths of the contracted elastic strands 208 in the elastomeric laminate 200. It is also to be appreciated that the apparatuses herein may include one of, some of, or all of adhesive applicator devices 334a, 334b, 334c and mechanical bonding device 336 mentioned herein.

It is also to be appreciated that the elastic strands 208 may be bonded with the first substrate 204 and/or second substrate 206 with various methods and apparatuses to create various elastomeric laminates, such as described in U.S. Patent Publication Nos. 2018/0168878 A1; 2018/0168877 A1; 2018/0168880 A1; 2018/0170027 A1; 2018/0169964 A1; 2018/0168879 A1; 2018/0170026 A1; 2018/0168889 A1; 2018/0168874 A1; 2018/0168875 A1; 2018/0168890 A1; 2018/0168887 A1; 2018/0168892 A1; 2018/0168876 A1; 2018/0168891 A1; 2019/0070042 A1; 2019/0070041 A1; 2021/0282797 A1; 2021/0275362 A1; 2022/0142828 A1; 2022/0362068 A1; 2024/0000619 A1; 2024/0000630 A1; 2024/0000631 A1; 2024/0000625 A1; 2024/0000624 A1; 2024/0000633 A1; 2024/0000632 A1; 2024/0000626 A1; 2024/0000634 A1; 2024/0000635 A1; 2024/0000637 A1; 2024/0000638 A1; 2024/0000627 A1, and combinations thereof, all of which are incorporated herein by reference.

It is to be appreciated that different components may be used to construct the elastomeric laminates 200 in accordance with the methods and apparatuses herein. For example, the first and/or second substrates 204, 206 may include nonwovens and/or films. In addition, the clastic strands 208 may be configured in various ways and may have various decitex values. In some configurations, the clastic strands 208 may be configured with decitex values ranging from about 10 decitex to about 1000 decitex, specifically reciting all 1 decitex increments within the above-recited range and all ranges formed therein or thereby.

As shown in FIGS. 5 and 6, the elastomeric laminate 200 may advance from the nip 328 and may be accumulated, such as for example, by being wound onto a roll 201R. As discussed in more detail below, the elastomeric laminate 200 may be wound onto a roll 201R in a fully extended state. The accumulated elastomeric laminate 200 may be stored and/or moved to a location for incorporation into an absorbent article assembly process, wherein the elastomeric laminate 200 may be converted into an absorbent article component, such as discussed above. As such, the accumulated elastomeric laminate 200 may be unwound from a roll 201R and incorporated into an absorbent article assembly line. It is to be appreciated that the apparatus 300a may be configured to assemble elastomeric laminates 200 that may be cut along the machine direction MD to define separate lanes of elastic of individual elastomeric laminates 200. In some configurations, the elastomeric laminate may be cut into separate lanes of individual elastomeric laminates 200 before wound onto respective rolls 201R. In some configurations, the elastomeric laminate may be cut into separate lanes of individual elastomeric laminates 200 as the elastomeric laminate is unwound from a roll 201R.

As previously mentioned, the apparatus 300a may include an unwinder 500 including spools 302 of elastic strands 208. It is to be appreciated the unwinder 500 may be configured with various quantities of spools 302 of elastic strands 208. Although FIG. 8 shows eighteen spools 302 positioned on the unwinder 500, and correspondingly, eighteen clastic strands 208 that may advance from the unwinder 500, it is to be appreciated that the unwinders 500 herein may be configured with more or less than eighteen spools 302 and more or less than eighteen elastic strands 208 advancing from the unwinder 500. In some configurations, the unwinders 500 herein may include from 1 to about 3000 spools 302 positioned thereon, and thus, may have from 1 to about 3000 clastic strands 208 advancing therefrom, specifically reciting all 1 spool and strand increments within the above-recited range and all ranges formed therein or thereby. In turn, the elastomeric laminates 200 herein may include from 1 to about 3000 elastic strands 208 spaced apart from each other in the cross direction CD, specifically reciting all 1 elastic strand increments within the above-recited range and all ranges formed therein or thereby.

It is also to be appreciated the unwinder 500 may be configured in various ways. For example, the unwinder 500 may be configured as a creel 502 adapted to support one or more spools 302 of clastic strands 208. FIG. 8 shows an example of an unwinder 500 that may include one or more mandrels 504 connected with a frame 506. It is to be appreciated that the frame 506 may be configured in various ways. For example, the frame 506 may include a first side 506a and a second side 506b connected with a base 506c. For the purposes of clarity, the first side 506a and the second side 506b are illustrated as being partially cut-away in FIG. 5. With continued reference to FIGS. 5, 6, and 8, the mandrels 504 may be rotatably connected with the frame 506 and may be adapted to rotate about a mandrel rotation axis 508. It is to be appreciated that the mandrels 504 may be oriented in various ways. For example, mandrels 504 may be horizontally or vertically oriented.

As shown in FIGS. 5, 6, 7, and 8, one or more spools 302 may be positioned on and supported by mandrels 504 of the unwinder 500. In some configurations, the cores 304 of one or more spools 302 may be adapted to receive and/or connect with the mandrel 504. As such, the spools 302 and the mandrel 504 may be adapted to rotate together. In some configurations, the mandrel 504 may be configured to drive and cause rotation of the spools 302. For example, FIG. 8 shows the mandrel 504 connected with a rotation driver 510, such as a motor or a servo motor, to drive and control the rotation of the mandrel 504. During operation, each spool 302 and the mandrel 504 are rotated in the same direction. An elastic strand 208 advances from the rotating spool 302 to downstream assembly operations, such as described herein. The unwinder 500 may also be configured such that the clastic strands 208 advance from the spools 302 at a speed S2 as described above. As clastic strands 208 are drawn from the rotating spools 302 supported on the mandrel 504, the outer diameter of the spools 302 become smaller. In turn, as the outer diameter of the spools 302 become smaller, the rotational speed of the mandrel 504 and spools 302 may need to increase in order to maintain a constant speed S2 of the elastic strands 208 advancing from the spools 302. As such, the apparatus 300a herein may include a sensor that detects the diameter of the spools 302, wherein feedback from the sensor can be used to control the speed of the rotation driver 510 and mandrel 504 to maintain a constant speed S2. In some configurations, the sensor may be configured to detect the tension in the elastic strands 208, wherein feedback from the sensor can be used to control the speed of the rotation driver 510 and/or mandrel 504 and/or mandrel 504 to maintain a desired tension in the strand 208.

As previously mentioned, one or more spools 302 may be positioned on and supported by the mandrel 504. And as shown in FIG. 8, the unwinder 500 may include one more mandrels 504 rotatably connected with a frame 506. It is to be appreciated that rotation drivers 510 may be directly connected with one or more mandrels 504 or indirectly connected with mandrels 504, such as through a transmission device, such as gear, pulley, chain, and/or belt arrangements. It is also to be appreciated that the mandrels 504 may be adapted to rotate independently of each other. In some configurations, the mandrels 504 may be rotationally connected with each other through a transmission device. It is to be appreciated that unwinder 500 may be connected with various arrangements of rotation drivers 510 adapted to rotate the mandrels 504 and/or spools 302 at the same or different speeds. For example, a plurality of mandrels 504 on the unwinder 500 may be connected with a single rotation driver 510 that may rotate the plurality of mandrels 504 and spools 302 thereon at the same speed. In another example, a plurality of mandrels 504 on the unwinder 500 may be connected with a single rotation driver 510 through a transmission device, and as such, may be configured to the drive the mandrels 504 and spools 302 thereon at the same or different speeds. In yet another example, a plurality of rotation drivers 510 may be configured to drive respective mandrels 504, each mandrel 504 having one or more spools 302 thereon. As such, the rotation drivers 510 may be configured to rotate respective mandrels 504 and spools 302 at the same or different speeds. In some configurations, spools 302 may be rotatably supported by the unwinder 500 without being driven, and as such, may be adapted to rotate as a result of respective clastic strands 208 being drawn therefrom.

It is also to be appreciated that one or more unwinders 500 and spools 302 of elastics 208 positioned thereon may be arranged along the cross direction CD of a converting process and/or arranged along a machine direction MD in various different portions of a converting process. For example, FIGS. 5 and 6 show an arrangement that includes a first unwinder 500a with first spools 302a of clastic strands 208a and a second unwinder 500b with second spools 302b of elastic strands 208b. The first and second clastic strands 208a, 208b may advance from the respective first and second unwinders 500a, 500b to be incorporated into the elastomeric laminate 200.

It is to be appreciated that the apparatuses and processes may be configured such that clastic strands 208 may be advanced from the unwinders 500 and directly to the assembly process without having to touch additional machine components, such as for example, guide rollers 514. It is also to be appreciated that in some configurations, elastic strands 208 may be advanced from the unwinders 500 and may be redirected and/or otherwise touched by and/or redirected by machine components, such as for example guide rollers 514, before advancing to the assembly process. Thus, it is to be appreciated that the first and/or second unwinders 500a, 500b and associated spools 302a, 302b may be arranged and/or oriented such that the rotation axes 508 of the mandrels 504 and/or rotation axes 308 of spools 302 may be parallel, perpendicular, or otherwise angularly offset with respect to the machine direction advancement of the elastomeric laminate 200 and/or the substrates 204, 206.

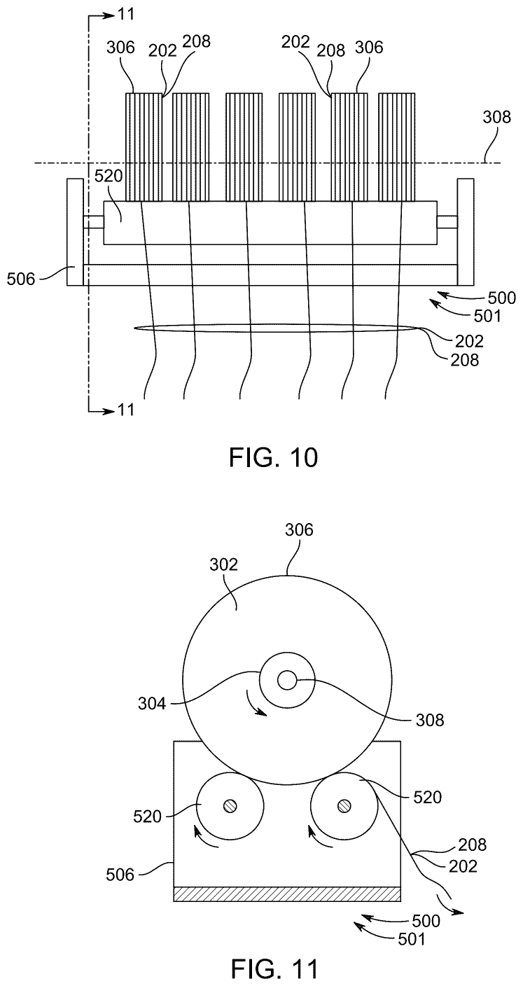

Although FIGS. 5, 6, and 8 illustrate unwinders 500 configured as mandrel driven unwinders, it is to be appreciated that the unwinders 500 herein may be configured in different ways. For example, the unwinders 500 may also be configured as surface driven unwinders 501, wherein the spools 302 may be driven by one or more rolls 520 in contact with the outer circumferential surfaces 306 of the spools 302, such as shown in FIGS. 10 and 11 and as disclosed in U.S. Patent Publication No. 2018/0170026 A1, which is incorporated by reference herein. It is to be appreciated that surface driven unwinders 501 may be configured to deliver elastic strands with varying strains by operating spools at different speeds and/or with spools having different diameters. It is also to be appreciated that surface driven unwinders 501 may also be configured to operate with spools 302 arranged in various ways, such as horizontal or vertical orientations. Different arrangements of spools 302 on unwinders 500 may be desirable for various reasons, such as for example, based on limited available space considerations. For example, surface driven unwinders may be configured to unwind elastic strands 208 from vertically arranged or stacked spools 302 with vertically oriented rotational axes 308, such as for example, available from Karl Mayer. Corporation.

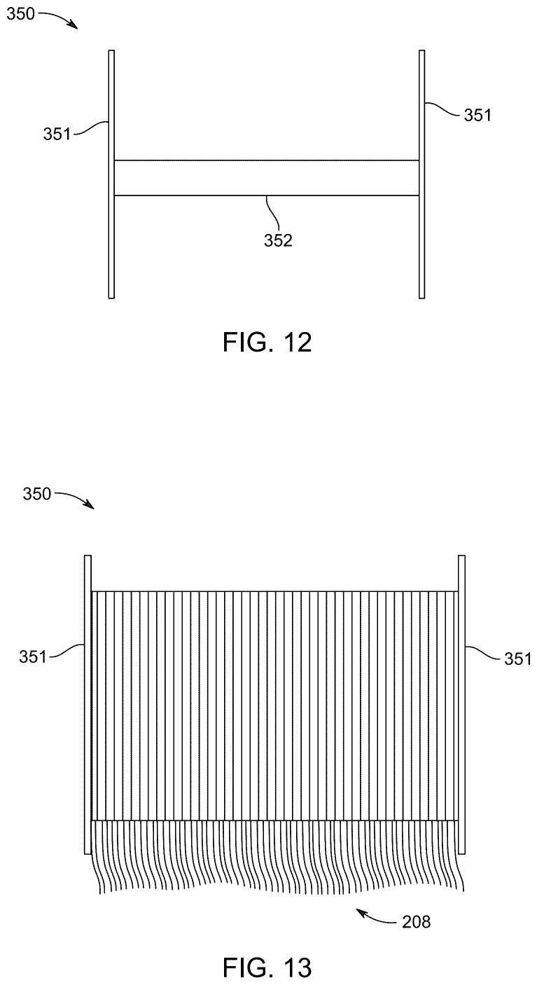

In addition, the apparatus 300a may be configured to assemble elastomeric laminates 200 with elastic strands 208 unwound from more than one unwinder 500 in combination with elastic strands supplied from various other types of elastic unwinder configurations, such as an overend unwinder and/or beams (also referred to as warp beams), such as disclosed in U.S. Pat. Nos. 6,676,054; 7,878,447; 7,905,446; 9,156,648; 4,525,905; 5,060,881; and 5,775,380; and U.S. Patent Publication No. 2004/0219854 A1, all of which are incorporated by reference herein. Additional examples of elastics and associated handling equipment are available from Karl Mayer Corporation. In some configurations, close cross directional spacing between low decitex elastic strands can be achieved by drawing such elastic strands that have been previously been wound onto a beam. FIG. 12 shows an example beam 350 that may include two side plates 351 connected with opposing ends of a mandrel core 352, and FIG. 13 shows an example of the beam of FIG. 12 with a plurality of strands 208 wound thereon.

In some configurations, the elastic strands 208 may include various types of spin finish, also referred herein as yarn finish, configured as coating on the elastic strands 208 that may be intended to help prevent the elastic strands from adhering to themselves, each other, and/or downstream handling equipment. In some configurations, a spin finish may include various types of oils and other components, such as disclosed for example in U.S. Pat. Nos. 8,377,554; 8,093,161; and 6,821,301, all of which are incorporated by reference herein. In some configurations, a spin finish may include various types of silicone oils, such as for example, polydimethylsiloxane. In some configurations, a spin finish may include various types of mineral oils, including hydrogenated paraffinic and napthenic oils. In some configurations, the molecular weight of an oil may be adjusted to optimize adhesion properties of the elastic strands depending on the process configuration in which the elastic strands may be used. In some configurations, a spin finish may include various types of fatty amides, erucamide, behenamide, and oleamide.

It is to be appreciated that the apparatuses 300a herein may be configured in various ways with various features described herein to assemble elastomeric laminates 200 having various stretch characteristics. For example, when the elastomeric laminate 200 is elongated, some elastic strands 208 may exert contraction forces in the machine direction MD that are different from contraction forces exerted by other elastic strands 208. Such differential stretch characteristics can be achieved by stretching some elastic strands 208 more or less than other elastic strands 208 before joining the elastic strands with the first and second substrates 204, 206. As discussed above, the spools 302 of elastic strands 208 may be unwound from one or more unwinders 500 at different speeds from each other, and as such, the elastic strands 208 may be stretched more or less than each when combined with the first and second substrates. For example, as previously discussed, the first substrate 204 and the second substrate 206 may each advance at a speed S1. In some configurations, the first elastic strands 208a may advance from first spools 302a at speed S2 that is less than the speed S1, and second elastic strands 208b may advance from second spools 302b at the speed S3 that is less than the speed S1. As such, the first elastic strands 208a and the second clastic strands 208b are stretched in the machine direction MD when combined with the first and second substrates 204, 206. In addition, the speed S2 may be less than or greater than the speed S3. Thus, the first elastic strands 208a may be stretched more or less than the second elastic strands 208b when combined with the first and second substrates 204, 206.

As discussed herein, the clastic strands 208 may be pre-strained prior to joining the clastic strands 208 to the first or second substrate layers 204, 206. In some configurations, the elastic strands 208 may be pre-strained from about 50% to about 300%, specifically reciting all 1% increments within the above-recited range and all ranges formed therein or thereby. In some configurations, the clastic strands 208 may be pre-strained from about 80% to about 250%, specifically reciting all 1% increments within the above-recited range and all ranges formed therein or thereby. Pre-strain refers to the strain imposed on an elastic or elastomeric material prior to combining it with another element of the elastomeric laminate or the absorbent article. Pre-strain is determined by the following equation: Pre-strain=((extended length of the clastic-relaxed length of the clastic)/relaxed length of the clastic)*100.

It is also to be appreciated that the elastic strands 208 may have various different material constructions and/or decitex values to create elastomeric laminates 200 having different stretch characteristics in different regions. In some configurations, the spools 302 of clastic strands 208 having different decitex values may be positioned on and advanced from one or more unwinders 500. In some configurations, the elastomeric laminate 200 may have regions where the clastic strands 208 are spaced relatively close to one another in the cross direction CD and other regions where the clastic strands 208 are spaced relatively far apart from each other in the cross direction CD to create different stretch characteristics in different regions. In some configurations, the clastic strands 208 may be supplied on the spool 302 in a stretched state, and as such, may not require additional stretching (or may require relatively less additional stretching) before being combined with the first substrate 204 and/or the second substrate 206. In some configurations, differential stretch characteristics in an elastomeric laminate 200 may be created by bonding another substrate and/or elastomeric laminate and/or an clastic film to a particular region of an elastomeric laminate. In some configurations, differential stretch characteristics in an elastomeric laminate 200 may be created by folding a portion of an elastomeric laminate onto itself in a particular region of the elastomeric laminate.

In some configurations, the clastic strands 208 may be joined with the first and second substrates 204, 206 such that the elastomeric laminate 200 may have different stretch characteristics in different regions along the cross direction CD, such as disclosed in U.S. Patent Publication Nos. 2006/0094319 A1; 2006/0032578 A1; 2018/0168878 A1; 2018/0168877 A1; 2018/0168880 A1; 2018/0170027 A1; 2018/0169964 A1; 2018/0168879 A1; 2018/0170026 A1; 2018/0168889 A1; 2018/0168874 A1; 2018/0168875 A1; 2018/0168890 A1; 2018/0168887 A1; 2018/0168892 A1; 2018/0168876 A1; 2018/0168891 A1; 2019/0070042 A1; and 2019/0070041 A1, which are all incorporated by reference herein. In some configurations, the elastomeric laminate 200 may include different tension zones that may help make some web handling operations less cumbersome, such as disclosed in U.S. Patent Publication No. 2002/0009940 A1, which is incorporated by reference herein.

It is also to be appreciated that the elastomeric laminate assembly operations herein may also be performed in conjunction with other operations. In some configurations, the elastomeric laminates 200 assembled with the methods and apparatuses herein may be subjected to various other manufacturing transformations before or after being accumulated. As discussed above, a continuous elastomeric laminate 200 may advance to a slitting operation, wherein the elastomeric laminate 200 is slit and separated along the machine direction MD into lanes, such as for example, a first continuous elastomeric laminate and a second continuous elastomeric laminate. It is to be appreciated that the elastomeric laminate 200 may be slit with a shear slitting operation or a crush slit operation. In a crush slit operation, the first substrate 204 and the second substrate 206 may be bonded together during the slitting operation. In some operations, the first and second substrates 204, 206 of an elastomeric laminate 200 may be bonded together along edges of the elastomeric laminate 200. For example, in some operations, edges of the first substrate 204 may be folded over opposing edge portions of the second substrate 206 to create sealed edges of the elastomeric laminate 200. It is to be appreciated that heat, pressure, adhesive, and/or ultrasonic bonding processes may be used to fixate such folded portions of the substrates. In some configurations, the locations of clastic strands 208 relative to side edges of elastomeric laminates 200 may be adjusted to change corrugation patterns along the side edges in desired manners. The elastomeric laminates 200 herein may be subject to additional operations to help provide aesthetic benefits, such as relatively more homogenous and/or consistent widths along the machine direction. In some configurations, edges of elastomeric laminates 200 may be trimmed to help improve aesthetics by providing relatively smooth and/or finished edges.

In some configurations, the first substrate 204 and/or the second substrate 206 may be subjected to aperturing processes during assembly operations of the elastomeric laminate 200. And in some configurations, the assembled elastomeric laminate 200 may be subjected to aperturing processes before or after being accumulated. It is to be appreciated that various different types of aperturing processes and operational configurations may be used, such as disclosed, for example, in U.S. Pat. No. 11,220,065, which is incorporated by reference herein. It is also to be appreciated that the first substrate 204, the second substrate 206, and/or the assembled elastomeric laminate 200 may be subjected to various other forming processes, such as embossing and others, such as disclosed, for example, in U.S. Patent Publication Nos. 2018/0228666 A1; 2018/0228656 A1; 2018/0228668 A1; 2019/0183689 A1; and 2018/0228669 A1, which are all incorporated by reference.

In some configurations, the first substrate 204 and/or the second substrate 206 may be subjected to printing operations during assembly operations of the elastomeric laminate 200. For example, print stations may be configured to print the first surface 210 and/or the second surface 212 of the first substrate 204 and/or to print the first surface 214 and/or the second surface 216 of the second substrate 206 before being combined to form the elastomeric laminate 200. In another example, print stations may be configured to print the first substrate 204 and/or to print the second substrate 206 after being combined to form the elastomeric laminate 200. It is to be appreciated that the printing stations may be configured in various ways and may include various types of printing accessories. For example, the printing stations may be capable of printing ink on substrate materials to form graphics by various printing methods, such as flexographic printing, rotogravure printing, screen-printing, inkjet printing, and the like. In some configurations, one or more lasers may be provided to create laser induced graphics on either or both the first substrate 204 and the second substrate 206.

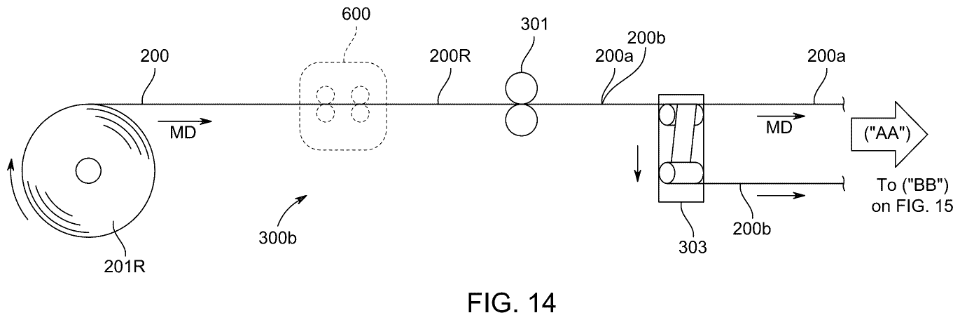

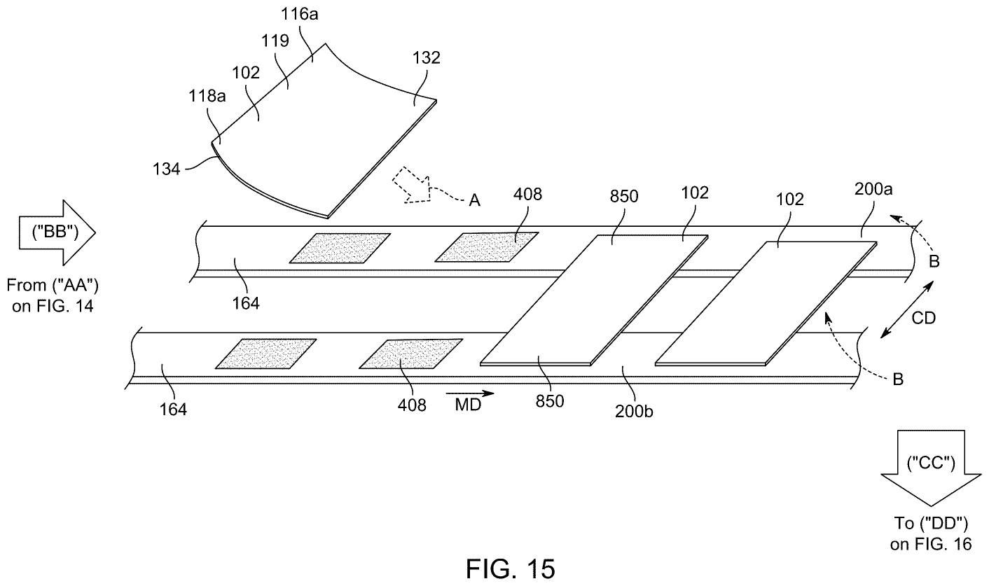

As discussed above, the assembled elastomeric laminate 200 may be accumulated, such as by being wound onto a roll 201R. In turn, the roll 201R may be transported to another location to be incorporated into an absorbent article assembly process. For example, as shown in FIG. 14, an absorbent article assembly apparatus 300b may be adapted to unwind the elastomeric laminate 200 from the roll 201R and advance the elastomeric laminate in a machine direction MD to be incorporated into an assembled absorbent article 100. The absorbent article assembly apparatus 300b may include a refreshing apparatus 600 generically represented by a dash line rectangle in FIG. 14. As discussed in more detail below, the refreshing apparatus may be configured to help restore stretch engine performance of the elastomeric laminate before incorporating the elastomeric laminate into an absorbent article assembly process. The refreshed elastomeric laminate 200R may advance in a machine direction MD from the refreshing apparatus 600 to the absorbent article assembly process. It is to be appreciated that some web handling/manufacturing operations may be completed upstream of, downstream from, or at the refreshing apparatus 600, such as tummy cutting operations that may create alternating low stretch zones and high stretch zones in the elastomeric laminate 200. In some configurations, cut lines may be formed through the elastomeric laminate to form perforations and various types of lines of weakness in the elastomeric laminate 200 upstream of, downstream from, or at the refreshing apparatus 600. It is also to be appreciated that the refreshed elastomeric laminate 200R may be combined with additional clastic material, substrates, and/or laminates as part of the absorbent article assembly process.

Referring again to FIG. 14, the refreshed elastomeric laminate 200R may advance from the refreshing apparatus 600 to a slitting station 301 that may be configured to cut the refreshed elastomeric laminate 200R along the machine direction MD to define separate lanes of individual refreshed elastomeric laminates 200R. For example, the slitting station 301 may comprise a slitting device that may comprise a knife and anvil that slits and separates the refreshed elastomeric laminate 200R along the machine direction MD into lanes, such as for example, a first elastomeric laminate 200a and a second elastomeric laminate 200b. It is to be appreciated slitting station 301 may be adapted to slit the elastomeric laminate 200 in various ways, such as for example with a shear slitting operation or a crush slit operation. In a crush slit operation, the first substrate 162 and the second substrate 164 may be bonded together during the slitting operation. It is to be appreciated that the slitting station 310 may be configured to perform slitting and/or cutting operations in various other ways, such as with lasers or ultrasonics, for example as disclosed in U.S. Patent Publication Nos. 2016/0354254 A1; 2016/0128874 A1; 2017/0266941 A1; 2017/0266057 A1; and 2017/0266056 A1, which are all incorporated by reference herein.

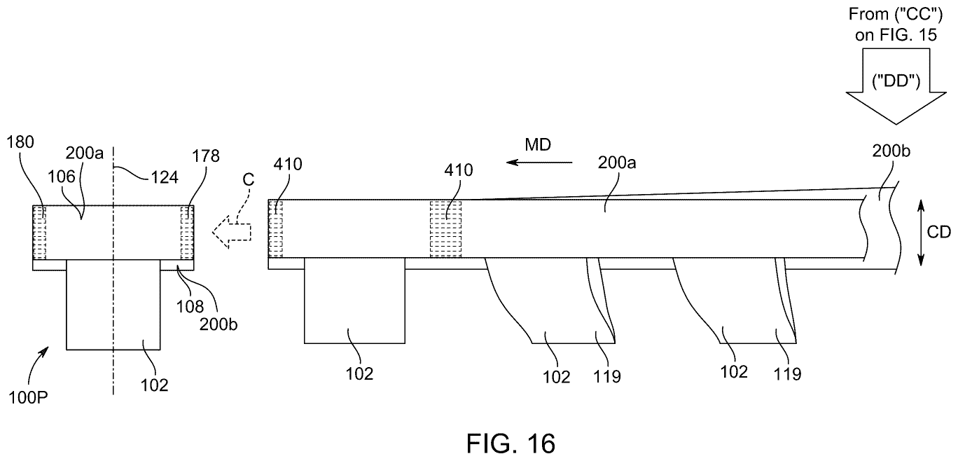

As shown in FIG. 14, the first and second elastomeric laminates 200a, 200b may advance from the slitting station 301 through a diverter 303 that separates the first and second elastomeric laminates 200a, 200b from each other in the cross direction CD. In some configurations, the diverter 303 may separate the first and second elastomeric laminates 200a, 200b in the cross direction CD to define a gap between the inner edge 107b of the first elastomeric laminate 200a and the inner edge 109b of the second elastomeric laminate 200b. As previously mentioned, the first elastomeric laminate 200a may correspond with the first elastic belt 106 and the second elastomeric laminate 200b may correspond with the second elastic belt 108 described above. As discussed in more detail with reference to FIGS. 15 and 16, when assembling diaper pants 100P, the first elastomeric laminate 200a and the second elastomeric laminate 200b may be separated from each other in the cross direction CD. In turn, opposing end regions of the chassis 102 may be connected with the first elastomeric laminate 200a and the second elastomeric laminate 200b. During subsequent assembly operations, the chassis 102 may be folded so as to position the first elastomeric laminate 200a into a facing relationship with the second elastomeric laminate 200b. The overlapping elastomeric laminates 200a, 200b may be bonded together, and subsequently, discrete diaper pants 100P may be formed by separating the first and second elastomeric laminates 200a, 200b into first and second belts 106, 108 by cutting along the cross direction CD through bonded regions of the first and second belt laminates 200a, 200b. As such, the bonded regions may be divided to define the first and second side seams 178, 180, respectively.

As shown in FIG. 15, the first elastic belt laminate 200a and the second elastic belt laminate 200b are separated from each other in the cross direction CD and adhesive 408 may be intermittently applied to first elastomeric laminate 200a and the second elastomeric laminate 200b. As shown in FIG. 15, adhesive 408 may be applied in a pattern to define adherence regions 410. It is to be appreciated that adhesive 408 may be applied in various ways and to define various shapes. Chassis 102 may be provided that comprise a body facing surface 132 and a garment facing surface 134, and an absorbent core 140 positioned between the body facing surface 132 and the garment facing surface 134 as discussed above. The chassis 102 may further comprise a first end region 116a and a second end region 118a separated in a cross direction CD from the first end region 116a by the crotch region 119. In turn, opposing end regions 116a, 118a of chassis 102 may be permanently bonded with the adhesive 408 in overlap regions 850 on the first elastomeric laminate 200a and/or a second elastomeric laminate 200b (represented by the dashed arrow “A”). In addition, the first end region 116a of the chassis 102 may be bonded with the first elastomeric laminate 200a, and the second end region 118a of the chassis 102 may be bonded with the second elastomeric laminate 200b.

During subsequent assembly operations shown in FIGS. 15 and 16, the chassis 102 may be folded in the crotch region 119 (represented by the dashed arrow “B”) so as to position the first elastomeric laminate 200a into a facing relationship with the second elastomeric laminate 200b as shown in FIG. 16. Bonds 410 may be applied to the overlapping belt laminates 200a, 200b. Subsequently, discrete diaper pants 100P may be formed by separating the first and second laminates 200a, 200b into first and second belts 106, 108 by cutting along the cross direction CD through the first and second laminates 200a, 200b adjacent the bonds 410 (represented by the dashed arrow “C”). As such, the bonds 410 may be divided to define the first and second side seams 178, 180, respectively.

It is to be appreciated that the elastomeric laminate 200 may be slit upstream or downstream of the apparatus 300. As discussed below, it is also to be appreciated that the refreshing apparatus 600 to refresh the elastomeric laminate 200 in various ways and may be positioned in various locations relative to other process operations, such as for example: chassis-belt combining operations; waist edge folding operations; side seaming operations; and final knife cutting operations.

As discussed above with reference to FIGS. 5-16, the elastomeric laminate 200 may advance from a laminate assembly process 300a to be accumulated, such as by winding the elastomeric laminate 200 onto a roll 201R. As discussed above with reference to FIG. 5, adhesive 218 may be applied to at least one of the elastic strands 208, the first substrate 204, and/or the second substrate 206 during the laminate assembly process. It is to be appreciated that the adhesive 218 may be a hot melt type of adhesive that is applied at an application temperature above an ambient temperature. Once the elastomeric laminate 200 is wound onto the roll 201R, the adhesive 218 in the elastomeric laminate 200 is allowed to cool from the application temperature to an ambient temperature. As such, the cooled adhesive 218 forms first discrete contiguous connections 700 between the elastic strands 208 and at least one of the first substrate 204 and the second substrate 206.

For the purposes of increased storage efficiency, the elastomeric laminate 200 may be wound on the roll 201R in a fully extended condition, essentially in a state where the elastomeric laminate 200 is extended to the extent that the corrugations allow the clastic strands 208 to elongate. In some configurations, the refreshing operations, mentioned above and described in more detail below, allow the elastomeric laminate 200 to be wound and stored on the roll 201R beyond a fully extended condition to further increase storage efficiency. For example, the first substrate 204 and/or the second substrate 206 may be stretched to a substrate stain value that is greater than zero when winding and storing the elastomeric laminate 200 onto the roll 201R. Here, the substrate strain value may be calculated as follows:

Substrate Strain Value (%)=100×(SL−RL)/(RL)

In the above equation, RL is a relaxed length of a substrate, and SL is a stretched length of the substrate. For example, a substrate having a relaxed length, RL, of 100 mm that is stretched to a stretched length SL of 103 mm has Substrate Strain Value of 103%. In some configurations, the elastomeric laminate 200 may be wound onto and stored on a roll 201R wherein the first substrate 204 and/or the second substrate 206 comprise a substrate strain value of 100% to about 104%, specifically reciting all 0.5% increments within the above-recited ranges and all ranges formed therein or thereby.

As discussed above, storing the elastomeric laminate 200 in a fully extended condition on a roll 201R may result in degraded stretch properties of the elastomeric laminate 200 manifested when the elastomeric laminate 200 is unwound from the roll 201R. It is to be appreciated that such degradation may also be exacerbated by winding and storing the elastomeric laminate 200 in a state wherein the first substrate 204 and/or the second substrate 206 comprises a substrate strain value that is greater than zero.

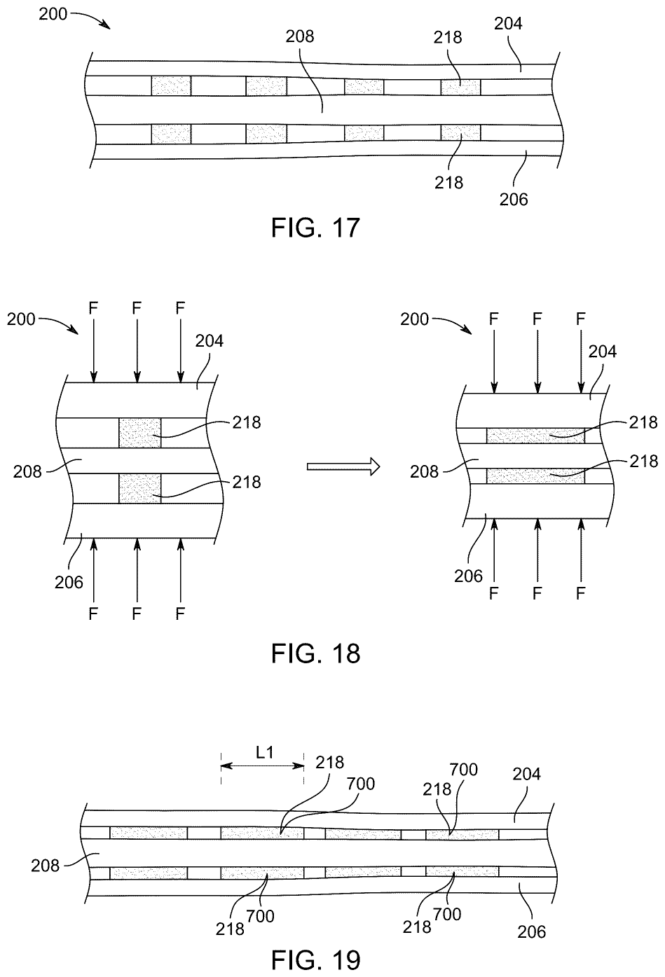

For example, as shown in FIG. 17, a schematic cross section view of the elastomeric laminate 200 is provided to illustrate adhesive 218 that has been applied during the laminate making process. As such, the adhesive in FIG. 17 may be at or near the application temperature. In a relatively short span of time (i.e., seconds or fractions of seconds), the assembled elastomeric laminate 200 may be wound onto the roll 201R while the adhesive 218 remains at an elevated application temperature. FIG. 18 illustrates how compressive forces F acting on the elastomeric laminate 200 resulting from winding compression on the roll 201R may cause the warm adhesive 218 to deform and progress lengthwise along the direction of stretch of the clastic strands 208. As the adhesive 218 in the elastomeric laminate 200 on the roll 201R cools to an ambient temperature, crystallizes, and sets, the adhesive 218 forms first discrete contiguous connections 700 between the clastic strands 208 and the first substrate 204 and/or the second substrate 206. FIG. 19 shows a cross sectional view of the elastomeric laminate 200 illustrating the first discrete contiguous connections 700 that may comprise first lengths L1 extending along a direction of stretch of the clastic strands 208. As such, the first lengths L1 may be relatively large due to the aforementioned deformation of the adhesive 218 while on the roll.

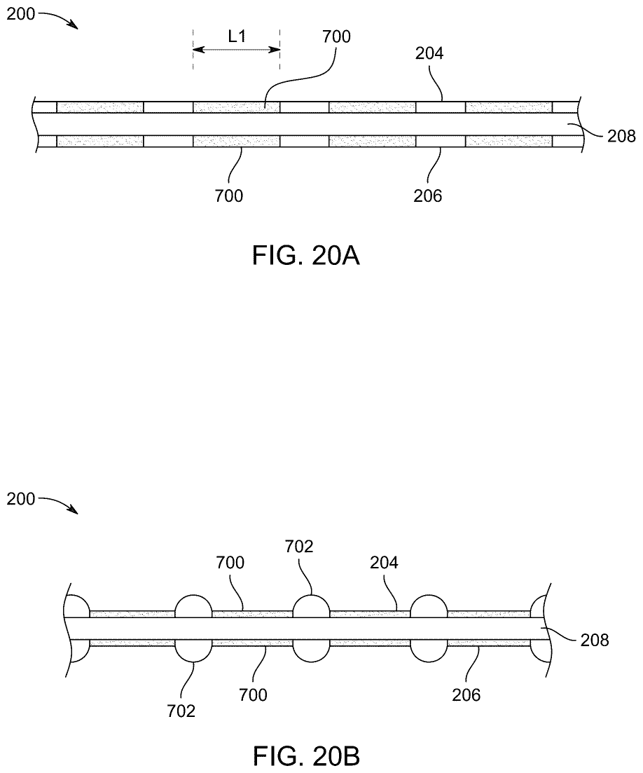

For example, FIG. 20A illustrates a cross-sectional view of an elastomeric laminate 200 in a fully extended state wherein the first substrate 204 and the second substrate 206 are bonded with an clastic strand 208 in a stretched state with first discrete contiguous connections 700 having first lengths L1. FIG. 20B illustrates a cross-sectional view of the elastomeric laminate 200 of FIG. 20A when the clastic strand 208 is allowed to contract. With particular reference to FIG. 20B, the clastic strand 208 contracts along portions of the first substrate 204 and/or the second substrate 206 that are not bonded with the clastic strand 208. As such, the contractive forces of the clastic strands 208 between first contiguous bonds 700 causes the first substrate 204 and/or second substrate 206 to buckle and/or separate from the clastic strand 208 during contraction and form corrugations 702. However, portions of the first substrate 204 and/or the second substrate 206 that are bonded with clastic strand 208 with the first contiguous bonds 700 are prevented from buckling and/or separating from the elastic strand 208, and therefore act to impede the ability of the elastic strand 208 to contract. In turn, relatively large first lengths L1 of first discrete contiguous connections 700 may compromise stretch engine performance of the elastomeric laminate 200. As a result, when the elastomeric laminate 200 is unwound from the roll 201R and incorporated into an assembled product, the first substate 204 and/or second substrate 206 resist contraction of the clastic strands 208 along the glued lengths of the first contiguous connections 700, which in turn, may reduce rugosity or gather formation of the elastomeric laminate 200 when incorporated into an assembled absorbent article 100.

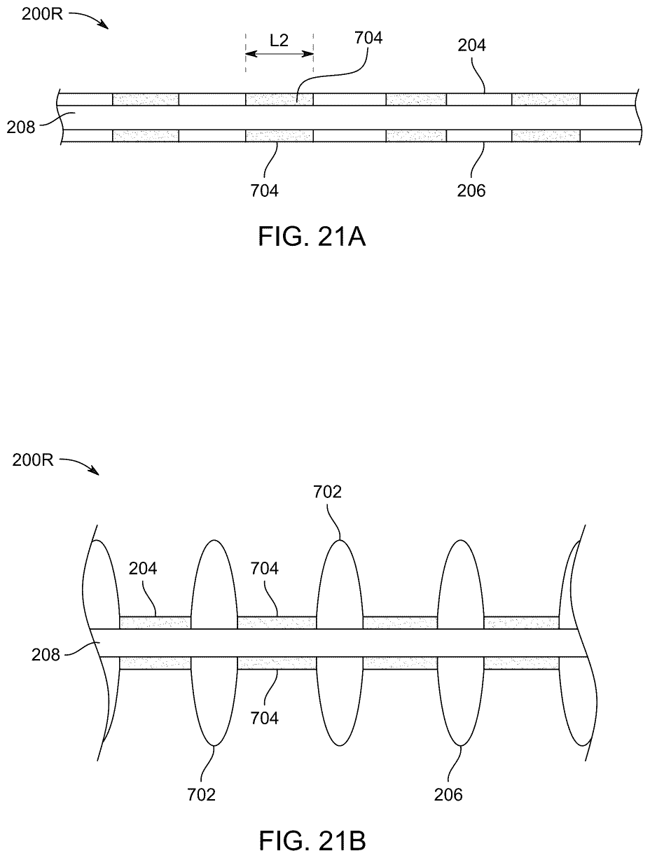

However, as mentioned above with reference to FIG. 14, according to processes herein, the elastomeric laminate 200 may be unwound and advanced from the roll 201R to a laminate refreshing apparatus 600 configured to restore stretch engine performance of the elastomeric laminate 200 before incorporating the refreshed elastomeric laminate 200R into an absorbent assembly process. As discussed above, the elastomeric laminate 200 may advance from the roll 201R in a machine direction MD with the adhesive 218 at an ambient temperature, wherein the clastic strands 208 are separated from each other in a cross direction CD. The refreshing apparatus 600 may be configured to apply localized tensions to the elastomeric laminate 200. As such, the elastomeric laminate 200 may be stretched in discrete locations such that strain values in the first substrate 204 and/or the second substrate 206 in the discrete locations increase from a first substrate strain value to a second substrate strain value that is greater than the first substrate strain value. In turn, application of the localized tensions operate to fracture and disrupt the first discrete contiguous connections 700 to form second discrete contiguous connections 704, such as shown in FIGS. 21A and 21B.

With reference to FIGS. 21A and 21B, the second discrete contiguous connections 704 comprise second lengths L2 that are less than the first lengths L1 of the first contiguous connections 700 discussed above. In turn, the clastic strands may contract along portions of the first substrate 204 and/or the second substrate 206 that are not bonded with elastic strand 208. As such, the contractive forces of the elastic strands 208 between second contiguous connections 704 causes the first substrate 204 and/or second substrate 206 to buckle and/or separate from the clastic strand 208 during contraction and form corrugations 702. Although the portions of the first substrate 204 and/or the second substrate 206 that are bonded with clastic strand with second contiguous connections 704 are prevented from buckling and/or separating from the elastic strand 208, the relatively smaller second lengths L2 of second discrete contiguous connections 704 compromise stretch engine performance of the refreshed elastomeric laminate 200R to a much lesser degree than the first contiguous connections 700 having the first lengths L1. The elastomeric laminate 200 may then advance from the refreshing apparatus 600 with the localized tensions removed from the elastomeric laminate to provide a refreshed elastomeric laminate 200R. The refreshed elastomeric laminate 200R may then advance to an article assembly process and may be converted into an absorbent article component.

As a result of the refreshing operations discussed herein, elastomeric laminates 200 according to methods herein may be wound onto and stored on a roll 201R while fully extended or with a first substrate 204 and/or second substate 206 having a substrate strain value greater than zero. The roll 201R may be stored and/or moved to a location for incorporation into an absorbent article manufacturing process. The elastomeric laminate 200 may then be unwound from the roll 201R and refreshed to have stretch engine performance restored before the refreshed elastomeric laminate 200R is converted into an absorbent article component.

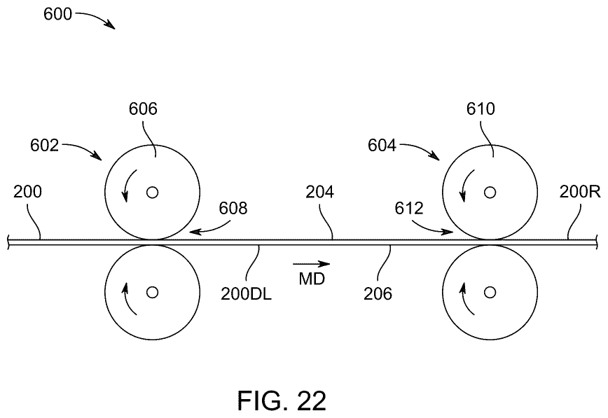

It is to be appreciated that the refreshing apparatuses 600 may be configured to apply localized tensions to the elastomeric laminate 200 in various ways. For example, FIG. 22 shows a refreshing apparatus 600 comprising a first web metering device 602 and a second web metering device 604 downstream from the first web metering device 602. The web first metering device 602 may comprise a first pair of rollers 606 arranged to define a first nip 608 therebetween, and the second web metering device 604 may comprise a second pair of rollers 610 arranged to define a second nip 612 therebetween. The first pair of rollers 606 may be configurated to rotate such that the elastomeric laminate 200 advances through the first nip 608 at a first speed S1, and the second pair of rollers 610 may be configured to rotate such that the elastomeric laminate 200 advances through the second nip 612 a second speed S2, wherein the second speed S2 is greater than the first speed S1. As such, the elastomeric laminate 200 is be stretched in the machine direction MD along discrete locations or lengths 200DL between the first nip 608 and the second nip 312 such that strain values in the first substrate 204 and/or the second substrate 206 in the discrete locations 200DL increase from a first substrate strain value to a second substrate strain value that is greater than the first substrate strain value. As discussed above, application of the localized tensions operate to fracture and disrupt the first discrete contiguous connections 700 to form second discrete contiguous connections 704. As the refreshed elastomeric laminate 200R advances from the second nip 612, the advancement speed of the elastomeric laminate may be reduced so that the strain values in the first substrate 204 and/or the second substrate 206 in the discrete locations 200DL may decrease from the second substrate strain value. It is to be appreciated that the first nip 608 and the second nip 612 may be positioned in close proximity to each other in the machine direction MD so as to apply localized tensions to a relative small length of the elastomeric laminate 200. In some configurations, the first nip 608 and the second nip 612 may be separated from each other so as to apply localized tensions to a length of elastomeric laminate 200DL of from about 50 mm to about 200 mm, specially reciting all 0.1 mm increments within the above-recited ranges and all ranges formed therein or thereby.

In some configurations, the refreshing apparatus 600 may be configured as intermeshing disks and/or gears that may apply localized tensions to the elastomeric laminate in the machine direction MD and/or the cross direction CD, such as disclosed for example in U.S. Pat. Nos. 7,824,594 and 7,896,641; and U.S. Patent Publication No. 2021/0282979/A1, which are incorporated by reference herein.

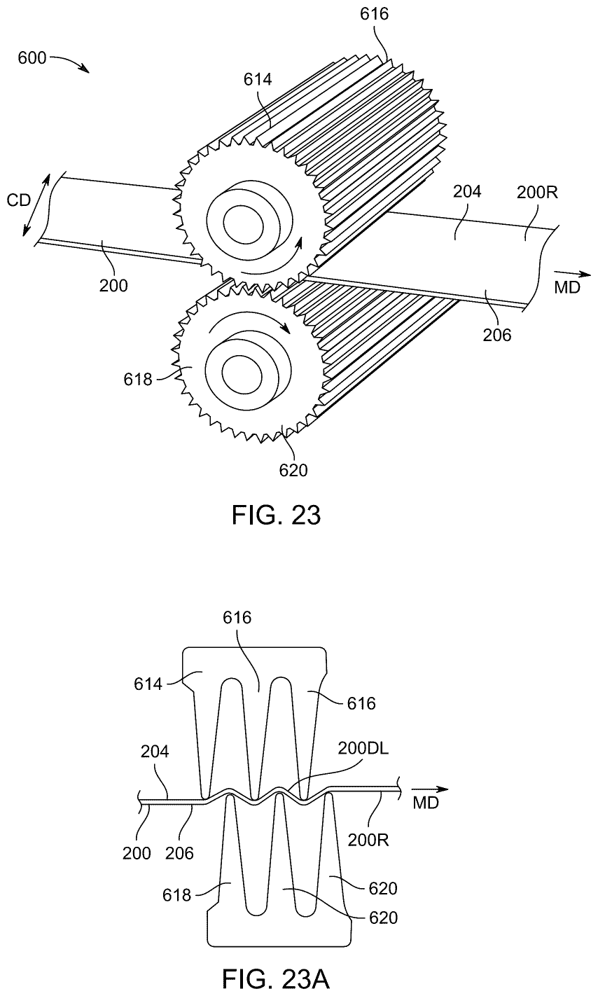

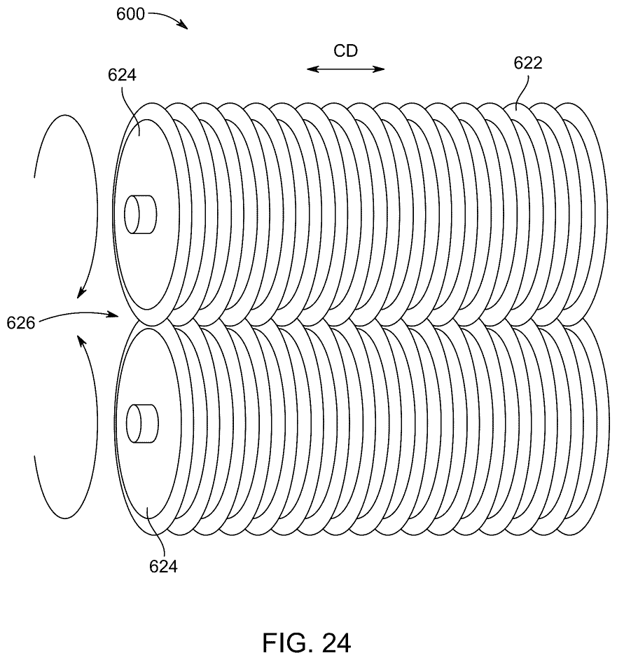

For example, FIG. 23 shows a refreshing apparatus 600 comprising a first gear 614 comprising first teeth 616 that extend in the cross direction CD and a second gear 618 with second teeth 620 that extend in the cross direction CD. The first gear 614 and the second gear 618 are arranged such that the first teeth 616 and the second teeth 620 intermesh with each other as the gears rotate. In operation, the elastomeric laminate 200 may advance in the machine direction MD between the first gear 614 and the second gear 618. As shown in FIG. 23A, the elastomeric laminate 200 may be stretched in the machine direction MD in discrete locations 200DL between intermeshing first teeth 616 and second teeth 620 such that strain values in the first substrate 204 and/or the second substrate 206 in the discrete locations 200DL increase from a first substrate strain value to a second substrate strain value that is greater than the first substrate strain value. As discussed above, the intermeshing first teeth 616 and second teeth 620 act to apply localized tensions to fracture and disrupt the first discrete contiguous connections 700 to form second discrete contiguous connections 704. As the refreshed elastomeric laminate advances from between the first and second gears 614, 618, the strain values in the first substrate 204 and/or the second substrate 206 in the discrete locations 200DL may decrease from the second substrate strain value. It is to be appreciated that various aspects of the gear designs may affect the strain levels imparted to the first substrate 204 and/or second substrate 206, such as for example, gear teeth shapes, tooth pitch, depth of teeth engagement, and/or the number of teeth on the gears.

In another example, FIG. 24 shows a refreshing apparatus 600 comprising a ring rolling apparatus 622, such as disclosed for example in U.S. Pat. Nos. 4,116,892; 4,834,741; 5,143,679; 5,156,793; 5,167,897; 5,422,172; and 5,518,801; and 9,687,580. In some configurations, the ring rolling apparatus 622 may include two profile rollers 624, such as shown for example in FIG. 24. It is to be appreciated that the rollers such as shown in FIG. 24 may be configured to be duplicate to each other. Each roller 624 may include a plurality of intermeshing disks that are situated on an axis. The elastomeric laminate 200 may advance through a nip 626 between the two profile rollers 624, and in turn, the ring rolling apparatus 622 may stretch the elastomeric laminate 200 in the cross direction CD in discrete locations between intermeshing disks such that strain values in the first substrate 204 and/or the second substrate 206 in the discrete locations increase from a first substrate strain value to a second substrate strain value that is greater than the first substrate strain value. As discussed above, the intermeshing disks act to apply localized tensions to disrupt the first discrete contiguous connections 700 to form second discrete contiguous connections 704. As the refreshed elastomeric laminate 200R advances from between the profiled rollers 624, the strain values in the first substrate 204 and/or the second substrate 206 in the discrete locations may decrease from the second substrate strain value.

In some configurations, the refreshing apparatuses 600 may be configured to apply heat to the elastomeric laminate 200 after being unwound from the roll 201R such that the adhesive 218 that forms the first connections 700 is heated such that the adhesive softens. As such, the first contiguous connections 700 between the elastic strands 208 and the first and/or second substrate 204, 206 may be reconfigured as to help restore have stretch engine performance of the elastomeric laminate 200R as the adhesive 218 in the refreshed elastomeric laminate 200R cools back to an ambient temperature, crystallizes, and sets.

As discussed above, reduced stretch engine performance may also be exacerbated by a relatively large winding compression of the elastomeric laminate 200 on the roll 201R. In some instances, excessively large winding pressures may squeeze the elastomeric laminate 200 near the center of the roll 201R and cause the elastomeric laminate 200 to partially telescope axially outward from the roll 201R. To help mitigate the likelihood of the “telescoping” phenomenon from occurring, in some configurations, the roll substrate strain value of the first substrate 204 and/or the second substrate 206 may be decreased during the winding process as a diameter of the roll 201R increases. In addition, the roll substrate strain value may be maintained above zero while winding. In other configurations, the likelihood of the “telescoping” phenomenon may also be reduced in some configurations when the elastomeric laminate 200 comprises elastic strands 208 having the same: decitex values; cross directional spacing; and pre-strain.

### Combinations

A1. A method for assembling absorbent articles, the method comprising steps of: unwinding an elastomeric laminate from a roll, wherein the elastomeric laminate comprises elastic strands bonded in a stretched state with a first substrate, wherein the first substrate on the roll comprises a roll substrate strain value that is greater than zero, the elastomeric laminate further comprising first discrete contiguous connections between the elastic strands and the first substrate formed by adhesive at an ambient temperature positioned between the elastic strands and the first substrate, the first discrete contiguous connections comprising first lengths extending along a direction of stretch of the elastic strands; advancing the elastomeric laminate from the roll in a machine direction, wherein the elastic strands are separated from each other in a cross direction; disrupting the first discrete contiguous connections to form second discrete contiguous connections by applying localized tensions to the elastomeric laminate to stretch the elastomeric laminate in discrete locations such that strain values in the first substrate in the discrete locations increase from a first substrate strain value to a second substrate strain value that is greater than the first substrate strain value, and wherein the second discrete contiguous connections comprise second lengths, wherein the second lengths are less than the first lengths, and removing the localized tensions from the elastomeric laminate to provide a refreshed elastomeric laminate; and converting the refreshed elastomeric laminate into an absorbent article component.
A2. The method of paragraph A1, wherein the step of converting the refreshed elastomeric laminate further comprises steps of: providing an absorbent chassis comprising a topsheet, a backsheet, and an absorbent core disposed between the topsheet and the backsheet, the absorbent chassis further comprising a first end region and an opposing second end region separated from each other by a central region, and having a longitudinal axis and a lateral axis; and bonding the first end region of absorbent chassis with the refreshed elastomeric laminate.
A3. The method of paragraph A2, further comprising a step of bonding a second elastomeric laminate with the second end region of the absorbent chassis.
A4. The method of paragraph A3, further comprising steps of: folding each chassis along the lateral axis to position the refreshed elastomeric laminate into a facing relationship with the second elastomeric laminate; and bonding the refreshed elastomeric laminate with the second elastomeric laminate to form pant diaper side seams; and cutting the refreshed elastomeric laminate into discrete pieces and allowing the elastic strands to contract.
A5. The method of any of paragraphs A1-A4, wherein the elastic strands comprise a decitex of from about 10 to about 200.
A6. The method of any of paragraphs A1-A5, wherein the elastic strands are separated from each other by about 0.5 mm to about 4 mm.
A7. The method of any of paragraphs A1-A6, wherein the elastic strands comprise a pre-strain of about 50% to about 300%.
A8. The method of any of paragraphs A1-A7, wherein the machine direction is parallel with the direction of stretch of the elastic strands.
A9. The method of any of paragraphs A1-A8, wherein the step of disrupting the first discrete contiguous connections further comprises applying localized tensions in the machine direction.
A10. The method of any of paragraphs A1-A9, wherein the step of disrupting the first discrete contiguous connections further comprises applying localized tensions in the cross direction.
A11. The method of any of paragraphs A1-A10, wherein the elastomeric laminate comprises elastic strands positioned in a stretched state between the first substrate and a second substrate.
A12. The method of paragraph A11, wherein first discrete contiguous connections between the elastic strands and the first substrate are also formed by the adhesive at an ambient temperature positioned between the elastic strands and the second substrate.
A13. The method of any of paragraphs A11-A12, wherein the first substrate comprises a first nonwoven and the second substrate comprises a second nonwoven.
A14. The method of any of paragraphs A11-A13, further comprising a step of providing the elastomeric laminate, the method comprising steps of: advancing the first substrate and the second substrate in a machine direction; advancing the elastic strands in the machine direction; separating neighboring elastic strands by a first distance from each other in a cross direction; stretching the elastic strands in the machine direction; applying adhesive to at least one of the elastic strands, the first substrate, and the second substrate, wherein the adhesive is applied at an application temperature above the ambient temperature; combining the stretched elastic strands with the first substrate and the second substrate to form an elastomeric laminate; stretching the elastomeric laminate such that at least one of the first substrate and the second substrate comprises the roll substrate strain value; winding the elastomeric laminate onto the roll with the at least one of the first substrate and the second substrate having the roll substrate strain value; and allowing the adhesive in the elastomeric laminate on the roll to cool to the ambient temperature and form the first discrete contiguous connections between the elastic strands and the first substrate.
A15. The method of paragraph A14, wherein the step of applying adhesive further comprises strand coating the adhesive onto the elastic strands.
A16. The method of any of paragraphs A14-A15, wherein the step of applying adhesive further comprises slot coating the adhesive onto at least one of the first substrate and the second
A17. The method of any of paragraphs A14-A16, wherein the step of applying adhesive further comprises meltblowing the adhesive onto at least one of the elastic strands, the first substrate, and the second substrate.
A18. The method of any of paragraphs A14-A17, further comprising a step of unwinding elastic strands from spools.
A19. The method of any of paragraphs A14-A18, wherein the first distance is about 0.5 mm to about 4 mm.
A20. The method of any of paragraphs A14-A19, wherein the step of winding the elastomeric laminate further comprises decreasing the roll substrate strain value as a diameter of the roll increases.
A21. The method of any of paragraphs A14-A20, wherein the step of stretching the elastic strands further comprises stretching the elastic strands to comprise a pre-strain of about 50% to about 300%.
B1. A method for supplying an elastomeric laminate, the method comprising steps of: advancing a first substrate in a machine direction; unwinding elastic strands from spools and advancing the elastic strands in the machine direction; separating neighboring elastic strands at a first distance from each other in a cross direction; stretching the elastic strands in the machine direction; applying adhesive to at least one of the elastic strands and the first substrate, wherein the adhesive is applied at an application temperature above an ambient temperature; forming an elastomeric laminate by combining the stretched elastic strands with the first substrate; stretching the first substrate to a strain value that is greater than zero; winding the elastomeric laminate onto the roll with the first substrate having the strain value that is greater than zero; and allowing the adhesive in the elastomeric laminate on the roll to cool to the ambient temperature and form first discrete contiguous connections between the elastic strands and the first substrate formed by the adhesive at the ambient temperature positioned between the elastic strands and the first substrate between the elastic strands and the first substrate, the first discrete contiguous connections comprising first lengths extending along a direction of stretch of the elastic strands.
B2. The method of paragraph B1, wherein the step of winding further comprises maintaining the strain value above zero while decreasing the strain value as a diameter of the roll increases.
B3. The method of any of paragraphs B1-B2, wherein the step of forming the elastomeric laminate further comprises combining the stretched elastic strands with the first substrate and a second substrate.

### Bio-Based Content for Components

Components of the absorbent articles described herein may at least partially be comprised of bio-based content as described in U.S. Pat. Appl. No. 2007/0219521 A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260™, SHE150™, or SGM9450F™ (all available from Braskem S. A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on Jun. 27, 2019.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for assembling absorbent articles, the method comprising steps of:
unwinding an elastomeric laminate from a roll, wherein the elastomeric laminate comprises elastic strands bonded in a stretched state with a first substrate, wherein the first substrate on the roll comprises a roll substrate strain value that is greater than zero, the elastomeric laminate further comprising first discrete contiguous connections between the elastic strands and the first substrate formed by adhesive at an ambient temperature positioned between the elastic strands and the first substrate, the first discrete contiguous connections comprising first lengths extending along a direction of stretch of the elastic strands;
advancing the elastomeric laminate from the roll in a machine direction, wherein the elastic strands are separated from each other in a cross direction;
disrupting the first discrete contiguous connections to form second discrete contiguous connections by applying localized tensions to the elastomeric laminate to stretch the elastomeric laminate in discrete locations such that strain values in the first substrate in the discrete locations increase from a first substrate strain value to a second substrate strain value that is greater than the first substrate strain value, and wherein the second discrete contiguous connections comprise second lengths, wherein the second lengths are less than the first lengths, and removing the localized tensions from the elastomeric laminate to provide a refreshed elastomeric laminate; and
converting the refreshed elastomeric laminate into an absorbent article component.

2. The method of claim 1, wherein the step of converting the refreshed elastomeric laminate further comprises steps of:
providing an absorbent chassis comprising a topsheet, a backsheet, and an absorbent core disposed between the topsheet and the backsheet, the absorbent chassis further comprising a first end region and an opposing second end region separated from each other by a central region, and having a longitudinal axis and a lateral axis; and
bonding the first end region of absorbent chassis with the refreshed elastomeric laminate.

3. The method of claim 2, further comprising a step of bonding a second elastomeric laminate with the second end region of the absorbent chassis.

4. The method of claim 3, further comprising steps of:
folding each chassis along the lateral axis to position the refreshed elastomeric laminate into a facing relationship with the second elastomeric laminate; and
bonding the refreshed elastomeric laminate with the second elastomeric laminate to form pant diaper side seams; and
cutting the refreshed elastomeric laminate into discrete pieces and allowing the elastic strands to contract.

5. The method of claim 1, wherein the elastic strands comprise a decitex of from about 10 to about 200.

6. The method of claim 1, wherein the elastic strands are separated from each other by about 0.5 mm to about 4 mm.

7. The method of claim 1, wherein the elastic strands comprise a pre-strain of about 50% to about 300%.

8. The method of claim 1, wherein the machine direction is parallel with the direction of stretch of the elastic strands.

9. The method of claim 1, wherein the step of disrupting the first discrete contiguous connections further comprises applying localized tensions in the machine direction.

10. The method of claim 1, wherein the step of disrupting the first discrete contiguous connections further comprises applying localized tensions in the cross direction.

11. The method of claim 1, wherein the elastomeric laminate comprises elastic strands positioned in a stretched state between the first substrate and a second substrate.

12. The method of claim 11, wherein first discrete contiguous connections between the elastic strands and the first substrate are also formed by the adhesive at an ambient temperature positioned between the elastic strands and the second substrate.

13. The method of claim 11, wherein the first substrate comprises a first nonwoven and the second substrate comprises a second nonwoven.

14. The method of claim 11, further comprising a step of providing the elastomeric laminate, the method comprising steps of:
advancing the first substrate and the second substrate in a machine direction;
advancing the elastic strands in the machine direction;
separating neighboring elastic strands by a first distance from each other in a cross direction;
stretching the elastic strands in the machine direction;
applying adhesive to at least one of the elastic strands, the first substrate, and the second substrate, wherein the adhesive is applied at an application temperature above the ambient temperature;
combining the stretched elastic strands with the first substrate and the second substrate to form an elastomeric laminate;
stretching the elastomeric laminate such that at least one of the first substrate and the second substrate comprises the roll substrate strain value;
winding the elastomeric laminate onto the roll with the at least one of the first substrate and the second substrate having the roll substrate strain value; and
allowing the adhesive in the elastomeric laminate on the roll to cool to the ambient temperature and form the first discrete contiguous connections between the elastic strands and the first substrate.

15. The method of claim 14, wherein the step of applying adhesive further comprises strand coating the adhesive onto the elastic strands.

16. The method of claim 14, wherein the step of applying adhesive further comprises slot coating the adhesive onto at least one of the first substrate and the second substrate.

17. The method of claim 14, wherein the step of applying adhesive further comprises meltblowing the adhesive onto at least one of the elastic strands, the first substrate, and the second substrate.

18. The method of claim 14, further comprising a step of unwinding elastic strands from spools.

19. The method of claim 14, wherein the first distance is about 0.5 mm to about 4 mm.

20. The method of claim 14, wherein the step of winding the elastomeric laminate further comprises changing the roll substrate strain value as a diameter of the roll increases.

21. The method of claim 20, wherein the step of winding the elastomeric laminate further comprises decreasing the roll substrate strain value as a diameter of the roll increases.

22. The method of claim 14, wherein the step of stretching the elastic strands further comprises stretching the elastic strands to comprise a pre-strain of about 50% to about 300%.

23. A method for supplying an elastomeric laminate, the method comprising steps of:
advancing a first substrate in a machine direction;
unwinding elastic strands from spools and advancing the elastic strands in the machine direction;
separating neighboring elastic strands at a first distance from each other in a cross direction;
stretching the elastic strands in the machine direction;
applying adhesive to at least one of the elastic strands and the first substrate, wherein the adhesive is applied at an application temperature above an ambient temperature;
forming an elastomeric laminate by combining the stretched elastic strands with the first substrate;
stretching the first substrate to a strain value that is greater than zero;
winding the elastomeric laminate onto the roll with the first substrate having the strain value that is greater than zero; and
allowing the adhesive in the elastomeric laminate on the roll to cool to the ambient temperature and form first discrete contiguous connections between the elastic strands and the first substrate formed by the adhesive at the ambient temperature positioned between the elastic strands and the first substrate between the elastic strands and the first substrate, the first discrete contiguous connections comprising first lengths extending along a direction of stretch of the elastic strands.

24. The method of claim 23, wherein the step of winding further comprises maintaining the strain value above zero while changing the strain value as a diameter of the roll increases.

25. The method of claim 24, wherein the step of winding further comprises maintaining the strain value above zero while decreasing the strain value as a diameter of the roll increases.

26. The method of claim 23, wherein the step of forming the elastomeric laminate further comprises combining the stretched elastic strands with the first substrate and a second substrate.
